SEQUENCE LISTING (26 sequences)

SEQ ID NO: 1 - protein, 33 aa, Artificial Sequence
  Peptide
  PEPTIDE (2)..(2): Gly, Ala, Aib, Sar
  PEPTIDE (5)..(5): Thr, Ser
  PEPTIDE (7)..(7): Thr, Ser
  PEPTIDE (8)..(8): Thr, Asp, Ser, Glu
  PEPTIDE (9)..(9): Asp, Glu
  PEPTIDE (10)..(10): Leu, Nle, Met, Val, Tyr
  PEPTIDE (12)..(12): Thr, Ser, Ala
  PEPTIDE (14)..(14): Leu, Nle, Met, Val
  PEPTIDE (15)..(15): Asp, Glu
  PEPTIDE (16)..(16): Ala, Asn, Gln, Gly, Ser, Glu, Asp, Arg, Lys
  PEPTIDE (19)..(19): Ala, Val, Leu
  PEPTIDE (20)..(20): Arg, Lys, His
  PEPTIDE (21)..(21): Asp, Glu
  PEPTIDE (24)..(24): Ala, Asn, Asp, Gln, Glu, Lys, Arg
  PEPTIDE (27)..(27): Ile, Leu, Val, Lys, Arg, Nle
  PEPTIDE (28)..(28): Gln, Asn, Lys, Arg
  PEPTIDE (29)..(29): Thr, Ser, Lys, Arg
  PEPTIDE (30)..(30): Lys, Arg
  PEPTIDE (31)..(31): Ile or absent
  PEPTIDE (32)..(32): Thr or absent
  PEPTIDE (33)..(33): Asp or absent
HXDGXFXXXXSXYXXXLAXXXFIXWLXXXXXXX

SEQ ID NO: 2 - protein, 34 aa, Artificial Sequence
  Peptide
  PEPTIDE (8)..(8): Thr, Asp, Ser, Glu
  PEPTIDE (10)..(10): Leu, Nle, Met, Val, Tyr
  PEPTIDE (16)..(16): Ala, Asn, Gln, Gly, Ser, Glu, Asp, Arg, Lys
  PEPTIDE (24)..(24): Ala, Asn, Asp, Gln, Glu, Lys, Arg
  PEPTIDE (31)..(31): Ile or absent
  PEPTIDE (32)..(32): Thr or absent
  PEPTIDE (33)..(33): Asp or absent
HGDGSFTXEXSTYLDXLAARDFIXWLIQTKXXXR

SEQ ID NO: 3 - protein, 31 aa, Artificial Sequence
  Peptide
HGDGSFTDELSTYLDALAARDFIAWLIQTKR

SEQ ID NO: 4 - protein, 31 aa, Artificial Sequence
  Peptide
HGDGSFTSELSTYLDALAARDFINWLIQTKR

SEQ ID NO: 5 - protein, 34 aa, Artificial Sequence
  Peptide
HGDGSFTDELSTYLDNLAARDFIAWLIQTKITDR

SEQ ID NO: 6 - protein, 34 aa, Artificial Sequence
  Peptide
HGDGSFTSELSTYLDALAARDFIAWLIQTKITDR

SEQ ID NO: 7 - protein, 34 aa, Artificial Sequence
  Peptide
HGDGSFTSELSTYLDNLAARDFINWLIQTKITDR

SEQ ID NO: 8 - protein, 32 aa, Artificial Sequence
  Peptide
RHGDGSFTSELSTYLDNLAARDFIAWLIQTKR

SEQ ID NO: 9 - protein, 31 aa, Artificial Sequence
  Peptide
HGDGSFTSELSTYLDALAARDFIAWLIQTKR

SEQ ID NO: 10 - protein, 34 aa, Artificial Sequence
  Peptide
HGDGSFTSELSTYLDNLAARDFIAWLIQTKITDR

SEQ ID NO: 11 - protein, 34 aa, Artificial Sequence
  Peptide
HGDGSFTDELSTYLDALAARDFIAWLIQTKITDR

SEQ ID NO: 12 - protein, 31 aa, Artificial Sequence
  Peptide
HGDGSFTDELSTYLDNLAARDFINWLIQTKR

SEQ ID NO: 13 - protein, 34 aa, Artificial Sequence
  Peptide
HGDGSFTDELSTYLDALAARDFINWLIQTKITDR

SEQ ID NO: 14 - protein, 34 aa, Artificial Sequence
  Peptide
HGDGSFTSELSTYLDALAARDFINWLIQTKITDR

SEQ ID NO: 15 - protein, 31 aa, Artificial Sequence
  Peptide
  PEPTIDE (10)..(10): Nle
HGDGSFTDEXSTYLDNLAARDFIAWLIQTKR

SEQ ID NO: 16 - protein, 34 aa, Artificial Sequence
  Peptide
  PEPTIDE (10)..(10): Nle
HGDGSFTDEXSTYLDNLAARDFIAWLIQTKITDR

SEQ ID NO: 17 - protein, 34 aa, Artificial Sequence
  Peptide
  PEPTIDE (10)..(10): Nle
HGDGSFTSEXSTYLDNLAARDFIAWLIQTKITDR

SEQ ID NO: 18 - protein, 34 aa, Artificial Sequence
  Peptide
  PEPTIDE (10)..(10): Nle
HGDGSFTSEXSTYLDALAARDFIAWLIQTKITDR

SEQ ID NO: 19 - protein, 33 aa, Artificial Sequence
  Peptide
HGDGSFKSELSTYLDALAARDFIAWLIQTKITD

SEQ ID NO: 20 - protein, 34 aa, Artificial Sequence
  Peptide
HGDGSFTKEELSTYLDALAARDFIAWLIQTKITD

SEQ ID NO: 21 - protein, 33 aa, Artificial Sequence
  Peptide
HGDGSFTSELKTYLDALAARDFIAWLIQTKITD

SEQ ID NO: 22 - protein, 33 aa, Artificial Sequence
  Peptide
HGDGSFTSELSKYLDALAARDFIAWLIQTKITD

SEQ ID NO: 23 - protein, 33 aa, Artificial Sequence
  Peptide
HGDGSFTSELSTYKDALAARDFIAWLIQTKITD

SEQ ID NO: 24 - protein, 33 aa, Artificial Sequence
  Peptide
HGDGSFTSELSTYLDKLAARDFIAWLIQTKITD

SEQ ID NO: 25 - protein, 33 aa, Artificial Sequence
  Peptide
HGDGSFTSELSTYLDAKAARDFIAWLIQTKITD

SEQ ID NO: 26 - protein, 33 aa, Artificial Sequence
  Peptide
HGDGSFTSELSTYLDALAAKDFIAWLIQTKITD

(19) United States
(12) Patent Application Publication
(10) Pub. No.: US 2018/0280480 A1
(43) Pub. Date: Oct. 4, 2018

(54) COMPOSITIONS AND PEPTIDES HAVING DUAL GLP-1R AND GLP-2R AGONIST ACTIVITY

(71) Applicant: GUBRA ApS, Hørsholm (DK)

(72) Inventors: Søren Ljungberg PEDERSEN, Borup (DK); Søren Blok Van WITTELOOSTUJN, København SV (DK); Pernille KONGSBAK-WISMANN, Copenhagen Ø (DK); Jacob JELSING, Roskilde (DK); Niels VRANG, Klampenborg (DK)

(73) Assignee: GUBRA ApS, Hørsholm (DK)

(21) Appl. No.: 15/521,908

(22) PCT Filed: Oct. 30, 2015
(86) PCT No.: PCT/EP2015/075292

§ 371 Date: Apr. 26, 2017

(30) Foreign Application Priority Data
Oct. 31, 2014 (EP) 14191274.1

(51) Int. Cl. A61K 38/26; A61P 3/10; A61K 45/06; A61P 1/00; A61P 31/12

(52) U.S. Cl. CPC A61K 38/26; A61P 3/10; A61P 31/12; A61P 1/00; A61K 45/06

(57) ABSTRACT

The present invention relates to compositions and peptides having agonist activity towards both the human GLP-1 receptor and the human GLP-2 receptor, wherein the relative agonist activity of the dual peptide towards the human GLP-1 receptor (GLP-1Rᵣₑₗₐₜᵢᵥₑ) is at least 0.01, and wherein the relative agonist activity of the dual peptide towards the human GLP-2 receptor (GLP-2rᵣₑₗₐₜᵢᵥₑ) is at least 0.01, and wherein (GLP-1Rᵣₑₗₐₜᵢᵥₑ)(GLP-2Rᵣₑₗₐₜᵢᵥₑ) is at least 0.01, as well as methods of production and uses thereof. Further, the invention relates to lipidated analogs of the peptides. The invention further relates to the treatment or prophylactic treatment of human diseases, in particular gut and brain relates diseases or metabolic disorders, such as gastrointestinal inflammation, short bowel syndrome and Crohn's disease.

### FIELD OF THE INVENTION

The present invention relates to compositions and peptides having dual GLP-1R and GLP-2R agonist activity and to the use thereof in preventing or treating human or animal disorders, such as bowel diseases leading to malabsorption and gut inflammation, such as short bowel syndrome or short bowel disease. Moreover, the compositions and dual GLP1R-GLP2R agonist peptides according to the invention are also useful for preventing or treating metabolic syndrome, obesity and diabetes. The invention further relates to pharmaceutical and veterinary compositions comprising the dual peptides, and to a method of production thereof.

### BACKGROUND OF THE INVENTION

Short bowel syndrome (SBS, also Short Gut Syndrome or simply Short Gut) is a malabsorption disorder, which may be caused by the surgical removal of parts of the small intestine or dysfunction of a segment of the bowel. Most disorders are caused by surgery, for example in connection with Crohn's disease, an inflammatory disorder of the digestive tract, volvulus, a spontaneous twisting of the small intestine that cuts off the blood supply and leads to tissue death, tumours of the small intestine, injury or trauma to the small intestine, necrotizing enterocolitis (premature newborn), bypass surgery to treat obesity, surgery to remove diseases or damaged portion of the small intestine. Further, some children are born with a congenital short bowel.

The symptoms of short bowel syndrome may include abdominal pain, diarrhoea, fluid depletion, weight loss and malnutrition. Patients with short bowel syndrome may have complications caused by malabsorption of vitamins and minerals, such as deficiencies in vitamins A, D, E, K, B9 (folic acid), and B12, calcium, magnesium, iron, and zinc. These may appear as anaemia, hyperkeratosis (scaling of the skin), easy bruising, muscle spasms, poor blood clotting, and bone pain.

There is no cure for short bowel syndrome except for transplants. However, several GLP-2 analogs have been suggested for the treatment of SBS.

Glucagon-like peptide-2 (GLP-2 or GLP2) is a 33-amino acid proglucagon-derived peptide produced by intestinal enteroendocrine cells. Native GLP-2 has the sequence H-HADGSFSDEMNTILDNLAARDFINWLIQTKITD-OH. Like glucagon-like peptide-1 (GLP-1 or GLP1) and glucagon itself, it is derived from the proglucagon peptide encoded by the GCG gene. GLP-2 stimulates intestinal growth and decreased enterocyte apoptosis. Moreover, GLP-2 prevents intestinal hypoplasia resulting from total parenteral nutrition. GLP-2R, a G protein-coupled receptor superfamily member is expressed in the gut and closely related to the glucagon receptor (GCGR) and the receptor for GLP-1 (GLP-1R). GLP-2 is the natural agonist for the GLP-2R. Once in the circulation, GLP-2 has a half-life of minutes, due to the rapid degradation by DPP-IV. The agent teduglutide, a glucagon-like peptide-2 analog developed by NPS Pharmaceuticals and Nycomed (Takeda), has been approved for use in the treatment of short bowel disease in the USA and in Europe and is marketed under the trademark Gattex/Revestive. Teduglutide is a DPP-IV resistant GLP-2 analog which results in improved pharmaco-dynamic properties.

It has further been suggested that GLP-1 has a positive effect on intestinal growth. Although the mechanism of this proposed action is not understood, it has been suggested that GLP-1 inhibits gastric motility and secretion, which may have positive effects.

The major forms of peptide glucagon-like peptide-1 (GLP-1 or GLP1) is GLP-1(7-37)-OH and GLP-1(7-36)-NH₂. They result from a selective cleavage from proglucagon. Active GLP-1 is secreted from L-cells in the gut subsequent to a meal. GLP-1 is a potent antihyperglycemic hormone, inducing glucose-dependent stimulation of insulin secretion while suppressing glucagon secretion. Native GLP-1 has the sequence H-HAEGTFTSDVSSYLEGQAAKEFIAWLVKG-OH. Once in the circulation, GLP-1 has a half-life of minutes, due to the rapid degradation by DPP-IV. GLP1 receptors (GLP-1R) are among other places expressed in the brain where they are involved in the control of appetite. It is well known that use of GLP-1 in a pharmaceutical can lead to reductions in food intake in mammals, consequently leading to a decrease in weight. Liraglutide is a long-acting glucagon-like peptide-1 agonist (GLP-1 agonist) developed by Novo Nordisk for the treatment of type 2 diabetes. Liraglutide is marketed under the brand name Victoza.

Both GLP-1 and GLP-2 are therefore believed to be involved in several gut and brain related diseases.

The aim of the present invention was to examine the effects of providing a combination treatment using simultaneous administration of peptide analogs providing GLP-1 and GLP-2 receptor activity.

Accordingly, it was an object of the present invention to investigate the suitability of compositions providing dual GLP-1 and GLP-2 receptor activity for use in the treatment of human or animal diseases, in particular in the treatment of gut and/or brain related diseases. More specifically, it was an object of the present invention to provide an improved treatment of gut and brain related diseases, in particular treatment of conditions related to short bowel disease.

It was a further object of the invention to provide single peptides having combined (dual) GLP-1 and GLP-2 receptor activity.

WO2013/164484 describes certain GLP-2 analogs with activity at both the GLP-1 and GLP-2 receptors. The receptor activity towards the GLP-1 and GLP-2 receptors is, however, not sufficient to be deemed dual agonist peptides according to the present invention. As apparent from Table 2 of WO2013/164484, the peptide having the greatest dual activity as defined in the present invention is “compound 11” having a relative GLP-1R activity of less than 0.03 and a relative GLP-2R activity of ⅓. (GLP-1Rᵣₑₗₐₜᵢᵥₑ) (GLP-2Rᵣₑₗₐₜᵢᵥₑ) of compound 11 is therefore less than 0.01.

It was a further object of the present invention to investigate the suitability of peptides having dual activity towards the GLP-1 and GLP-2 receptors for use in the treatment of human or animal diseases, in particular in the treatment of gut and/or brain related diseases, more particularly treatment of conditions related to short bowel disease.

### SUMMARY OF THE INVENTION

The present invention is based on the surprising finding that compositions providing dual GLP-1 and GLP-2 receptor activity are suitable for use in the treatment of human or animal diseases, in particular in the treatment of gut and/or brain related diseases.

As seen in the experimental part of the present application, the combined treatment with a GLP-1 and a GLP-2 analog surprisingly resulted in a significant and synergistic increase in gut weight or volume, while simultaneously providing a decreasing effect on food intake in mice and rats compared to the other treatment groups.

The present invention is further based on the surprising identification and development of peptide analogs having dual agonist functions against both the GLP-1 and GLP-2 receptors.

The invention is further based on the surprising finding of an increased efficacy of these dual agonists in treating certain gut and brain related diseases.

Accordingly, in a first aspect, the invention relates to pharmaceutical compositions comprising a first peptide or a lipidated analog thereof, said first peptide providing agonist activity towards the human GLP-1 receptor and a second peptide or a lipidated analog thereof, said second peptide providing agonist activity towards the human GLP-2 receptor, wherein the relative agonist activity of the peptides towards the human GLP-1 receptor (GLP-1Rᵣₑₗₐₜᵢᵥₑ) is at least 0.01, and wherein the relative agonist activity of the peptides towards the human GLP-2 receptor (GLP-2Rᵣₑₗₐₜᵢᵥₑ) is at least 0.01, and wherein (GLP-1Rᵣₑₗₐₜᵢᵥₑ)(GLP-2Rᵣₑₗₐₜᵢᵥₑ) is at least 0.01, for use in the induction of gut proliferation.

In a further aspect, the present invention relates to a pharmaceutical composition comprising a first peptide or a lipidated analog thereof, said first peptide providing agonist activity towards the human GLP-1 receptor and a second peptide or a lipidated analog thereof, said second peptide providing agonist activity towards the human GLP-2 receptor, wherein the relative agonist activity of the peptides towards the human GLP-1 receptor (GLP-1Rᵣₑₗₐₜᵢᵥₑ) is at least 0.01, and wherein the relative agonist activity of the peptides towards the human GLP-2 receptor (GLP-2Rᵣₑₗₐₜᵢᵥₑ) is at least 0.01, and wherein the peptides provides a (GLP-2Rᵣₑₗₐₜᵢᵥₑ)>(GLP-1Rᵣₑₗₐₜᵢᵥₑ).

In a highly preferred aspect of the invention, the peptides designated above as the “first” peptide and the “second” peptide are the same peptides.

Accordingly, in a highly preferred aspect, the present invention relates to a dual peptide having agonist activity towards the human GLP-1 receptor and the human GLP-2 receptor, wherein the relative agonist activity of the dual peptide towards the human GLP-1 receptor (GLP-1Rᵣₑₗₐₜᵢᵥₑ) is at least 0.01, and wherein the relative agonist activity of the dual peptide towards the human GLP-2 receptor (GLP-2Rᵣₑₗₐₜᵢᵥₑ) is at least 0.01, and wherein (GLP-1Rᵣₑₗₐₜᵢᵥₑ)(GLP-2Rᵣₑₗₐₜᵢᵥₑ) is at least 0.01, a pharmaceutically acceptable salt, solvate or lipidated analog thereof.

In a further aspect, the invention relates to the use of such compositions and dual peptides for the treatment of a human or animal subject. In a further aspect thereof, the invention relates to the use of such compositions and dual peptides for treatment of short bowel disease in a human or animal subject.

In another aspect, the invention relates to a peptide comprising an amino acid sequence according to SEQ ID NO: 1 or a lipidated peptide analog having no more than 2 deviations in the amino acid sequence of SEQ ID NO: 1;

(SEQ ID NO: 1)
H-X₂-D-G-X₅-F-X₇-X₈-X₉-X₁₀-S-X₁₂-Y-X₁₄-X₁₅-X₁₆-L-A-X₁₉-
X₂₀-X₂₁-F-I-X₂₄-W-L-X₂₇-X₂₈-X₂₉-X₃₀-X₃₁-X₃₂-X₃₃,

wherein X₂ is Gly, Ala, Aib (2-aminoisobutyric acid) or Sar (N-methyl glycin); X₅ is Thr or Ser; X₇ is Thr or Ser; X₈ is Thr, Asp, Ser or Glu; X₉ is Asp or Glu; X₁₀ is Leu, Nle (Norleucine), Met, Val or Tyr; X₁₂ is Thr, Ser or Ala; X₁₄ is Leu, Nle (Norleucine), Met or Val; X₁₅ is Asp or Glu; X₁₆ is Ala, Asn, Gln, Gly, Ser, Glu, Asp, Arg or Lys; X₁₉ is Ala, Val or Leu; X₂₀ is Arg, Lys or His; X₂₁ is Asp or Glu; X₂₄ is Ala, Asn, Asp, Gln, Glu, Lys or Arg; X₂₇ is Ile, Leu, Val, Lys, Arg or Nle (Norleucine); X₂₈ is Gln, Asn, Lys or Arg; X₂₉ is Thr, Ser, Lys or Arg; X₃₀ is Lys or Arg; X₃₁ is Ile or absent; X₃₂ is Thr or absent; X₃₃ is Asp or absent.

In a further aspect, the invention relates to the use of such peptides for the treatment of a human or animal subject. In a further aspect, the invention relates to the use of such peptides for treatment of gut and brain related diseases in a human or animal subject. In a further aspect, the invention relates to the use of such peptides for the treatment of short bowel disease in a human or animal subject.

In yet another aspect, the invention relates to a pharmaceutical or veterinary composition comprising a dual agonist peptide according to the invention and at least one pharmaceutical or veterinary excipient.

In yet another aspect, the invention relates to a nucleic acid molecule comprising a nucleic acid sequence encoding the peptide according to the invention as well as to a method of producing a peptide according to the invention.

### Definitions

In the context of the present invention, “peptide” means a chain of amino acid monomers (termed “residues”) linked by peptide (amide) bonds. Peptides according to the invention have an N-terminal and a C-terminal amino acid residue at each of the ends of the peptide. The N-terminal amino acid residue preferably comprises an R₁-group being selected among hydrogen, methyl, acetyl, formyl, benzoyl, trifluoroacetyl, and the C-terminal amino acid residue preferably comprises an R₂ group being NH₂ or OH. According to the invention, the term “peptide” includes pharmaceutically acceptable salts or solvates of the chain(s) of amino acid monomers.

In the context of the present invention, “agonist activity towards the human GLP-1 receptor” means the ability to activate the GLP-1 receptor, as determined by appropriate in vitro assays, for example as described in the experimental part below.

In the context of the present invention, the “relative agonist activity of the dual peptide towards the human GLP-1 receptor” (GLP-1Rᵣₑₗₐₜᵢᵥₑ) is a measure of the relative activity towards the GLP-1 receptor, measured as EC₅₀-concentrations, of the relevant compound or dual peptide compared with the corresponding activity, measured as EC₅₀-concentrations, of the native GLP-1 measured under identical conditions. The EC₅₀ value is a measure of the concentration required to achieve half of the maximal activity in a particular assay. If a peptide has an EC₅₀ at a particular receptor (e.g. GLP-1R) which is lower than the EC₅₀ of a reference peptide (in the same assay), the peptide has a higher potency at that receptor than the reference peptide. As an example, if the native GLP-1 (reference) provides an EC₅₀ of 0.01 nM in an appropriate assay, and the dual peptide provides an EC₅₀ of 0.02 nM under identical conditions, the relative activity of the dual peptide (GLP-1Rᵣₑₗₐₜᵢᵥₑ) is 0.01 nM/0.02 nM=0.5 or 50%.

In the context of the present invention, “agonist activity towards the human GLP-2 receptor” means the ability to activate the GLP-2 receptor, as determined by appropriate in vitro GLP-1 assays, for example as described in the experimental part below.

In the context of the present invention, the “relative agonist activity of the dual peptide towards the human GLP-2 receptor” (GLP-2Rᵣₑₗₐₜᵢᵥₑ) is a measure of the relative activity towards the GLP-2 receptor, measured as EC₅₀-concentrations, of the relevant compound or dual peptide compared with the corresponding activity, measured as EC₅₀-concentrations, of the native GLP-2 measured under identical conditions. As an example, if the native GLP-2 provides an EC₅₀ of 0.01 nM in an appropriate assay, and the dual peptide provides an EC₅₀ of 0.10 nM under identical conditions, the relative activity of the dual peptide (GLP-1Rᵣₑₗₐₜᵢᵥₑ) is 0.01 nM/0.10 nM=0.1 or 10%.

In the context of the present invention, “treatment” is intended to cover medical treatment and prophylactic treatment of one or more undesired condition(s) occurring or potentially occurring in a subject. As such, the undesired condition does not necessarily need to be clinically classified or classifiable as a disease or as a condition for which treatment is clinically needed. The subject suffering from an undesired condition is preferably a mammal. Even more preferably, the subject suffering from an undesired condition is a human.

In the context of the present invention, “Serum albumin binding amino acid” means an amino acid such as a natural or non-natural amino acid which has been modified by attaching a serum albumin binding side chain to the amino acid backbone. The attachment of a serum albumin binding side chain to the amino acid backbone may also be termed “lipidation”.

In the context of the present invention, “Serum albumin binding side chain” means a side chain attached to an amino acid (residue), the side chain consisting of or comprising a functional group capable of binding to serum albumin. A functional group is capable of binding to serum albumin according to the present invention if the side chain result in an increased binding affinity in an albumin binding biocore assay compared to a non lipidated peptide. Another example of a method for determination of albumin binding is by measuring the binding of the peptide using enzyme-linked immunosorbent assay (ELISA) using a microtiter plate with immobilised albumin as described in US 20110275559 A1. An alternative but indirect method to identify derivatives with high albumin affinity is to pick those analogs that had a right shift of the binding dose-response curve when high concentrations of albumin were in the assay compared to binding at low albumin concentration (Lau et al. 2015, Journal of Medicinal Chemistry 58 (18): 7370-7380). In a preferred embodiment of the present invention, the serum albumin binding side chain comprises a lipid molecule (termed lipidated amino acid), cholesterol molecule or sialic acid molecules.

In the context of the present invention, “lipidated analog” means an analog of a peptide having a sequence as defined in the claims. The analogs according to the invention are analogs wherein the sequence (as defined in the claims) has been altered (and therefore may deviate) by the introduction (e.g. by substitution) of one or more, preferably only one, serum albumin binding amino acid residue(s) and one or more serum albumin binding side chain(s).

### DETAILED DESCRIPTION OF THE INVENTION

During the experiments leading to the present invention, it was surprisingly found that compositions providing dual activity towards the GLP-1 and GLP-2 receptors resulted in decreased food intake in mice, compared to the other treatment groups, while simultaneously providing a significant and synergistic increase in gut weight.

Thus, it was concluded that such compositions are suitable for use in the treatment of certain human or animal diseases, in particular in the treatment of gut and/or brain related diseases.

It was further surprisingly found possible to develop dual peptide agonists having activity towards both the GLP-1 receptor and the GLP-2 receptor.

Accordingly, the present invention relates to compositions and to peptides having dual GLP-1 and GLP-2 receptor activity or lipidated analogs thereof, wherein the relative agonist activity towards the human GLP-1 receptor (GLP-1Rᵣₑₗₐₜᵢᵥₑ) is at least 0.01, and wherein the relative agonist activity of the compound towards the human GLP-2 receptor (GLP-2Rᵣₑₗₐₜᵢᵥₑ) is at least 0.01, and wherein (GLP-1Rᵣₑₗₐₜᵢᵥₑ)(GLP-2Rᵣₑₗₐₜᵢᵥₑ) is at least 0.01 for use in the induction of gut proliferation.

Further, the invention relates to a pharmaceutical composition comprising a first peptide or a lipidated analog thereof, said first peptide providing agonist activity towards the human GLP-1 receptor and a second peptide or a lipidated analog thereof, said second peptide providing agonist activity towards the human GLP-2 receptor, wherein the relative agonist activity of the peptides towards the human GLP-1 receptor (GLP-1Rᵣₑₗₐₜᵢᵥₑ) is at least 0.01, and wherein the relative agonist activity of the peptides towards the human GLP-2 receptor (GLP-2Rᵣₑₗₐₜᵢᵥₑ) is at least 0.01, and wherein the peptides provides a (GLP-2Rᵣₑₗₐₜᵢᵥₑ)>(GLP-1Rᵣₑₗₐₜᵢᵥₑ). In a preferred aspect thereof, the first peptide and the second peptide are the same (dual) peptide being both a GLP-1 acting compound and a GLP-2 acting compound.

In a preferred aspect thereof, the GLP-2 acting compound in the composition has a GLP-2Rᵣₑₗₐₜᵢᵥₑ that is at least 0.1, preferably at least 0.2, more preferably at least 0.3, even more preferably at least 1.

In a more preferred aspect, the compounds in the composition have a relative activity wherein (GLP-1Rᵣₑₗₐₜᵢᵥₑ)(GLP-2Rᵣₑₗₐₜᵢᵥₑ) is at least 0.02, preferably at least 0.03, more preferably at least 0.2, even more preferably at least 0.5.

In an even more preferred aspect thereof, the compounds in the composition have a relative activity wherein (GLP-2Rᵣₑₗₐₜᵢᵥₑ)>(GLP-1Rᵣₑₗₐₜᵢᵥₑ), preferably wherein (GLP-2Rᵣₑₗₐₜᵢᵥₑ)>2(GLP-1Rᵣₑₗₐₜᵢᵥₑ), even more preferably wherein (GLP-2Rᵣₑₗₐₜᵢᵥₑ)>10(GLP-1Rᵣₑₗₐₜᵢᵥₑ).

The GLP-1 analog liraglutide has a relative GLP-1 activity of 0.1. The GLP-2 analog teduglutide has a relative GLP-2 activity of 1.0.

In a preferred embodiment, the invention relates to a peptide having dual agonist activity towards the human GLP-1 receptor and the human GLP-2 receptor, wherein the relative agonist activity of the dual peptide towards the human GLP-1 receptor (GLP-1Rᵣₑₗₐₜᵢᵥₑ) is at least 0.01, and wherein the relative agonist activity of the dual peptide towards the human GLP-2 receptor (GLP-2Rᵣₑₗₐₜᵢᵥₑ) is at least 0.01, and wherein (GLP-1Rᵣₑₗₐₜᵢᵥₑ)(GLP-2Rᵣₑₗₐₜᵢᵥₑ) is at least 0.01. The invention further relates to lipidated analogs of such peptides.

The invention further relates to pharmaceutically acceptable salts and solvates thereof.

The dual activity of the peptides according to the invention is the activity towards the human GLP-1 receptor and the human GLP-2 receptor, wherein the relative agonist activity of the dual peptide towards the human GLP-1 receptor (GLP-1Rᵣₑₗₐₜᵢᵥₑ) is at least 0.01, and wherein the relative agonist activity of the dual peptide towards the human GLP-2 receptor (GLP-2Rᵣₑₗₐₜᵢᵥₑ) is at least 0.01, and wherein (GLP-1Rᵣₑₗₐₜᵢᵥₑ) (GLP-2Rᵣₑₗₐₜᵢᵥₑ) is at least 0.01, or a pharmaceutically acceptable salt or solvate thereof.

The EC₅₀ of native GLP-1 towards the GLP-1R is (in most assays) lower than the EC₅₀ of native GLP-2 towards the GLP-2R. Therefore, in certain aspects of the invention, it is preferred to have a relatively high GLP-2 activity. In a preferred aspect of the invention, GLP-2Rᵣₑₗₐₜᵢᵥₑ is at least 0.1. In a more preferred aspect of the invention, GLP-2Rᵣₑₗₐₜᵢᵥₑ is at least 0.2. In an even more preferred aspect of the invention, GLP-2Rᵣₑₗₐₜᵢᵥₑ is at least 0.3. In an even more preferred aspect of the invention, GLP-2Rᵣₑₗₐₜᵢᵥₑ is at least 1.0.

It is further preferred that the peptides according to the invention have a real dual activity, i.e. the combined activity is as high as possible. Thus, in a preferred aspect of the invention, (GLP-1Rᵣₑₗₐₜᵢᵥₑ) (GLP-2Rᵣₑₗₐₜᵢᵥₑ) is at least 0.02. In a more preferred aspect of the invention, (GLP-1Rᵣₑₗₐₜᵢᵥₑ) (GLP-2Rᵣₑₗₐₜᵢᵥₑ) is at least 0.03. In an even more preferred aspect of the invention, (GLP-1Rᵣₑₗₐₜᵢᵥₑ) (GLP-2Rᵣₑₗₐₜᵢᵥₑ) is at least 0.2. In an even more preferred aspect of the invention, (GLP-1Rᵣₑₗₐₜᵢᵥₑ) (GLP-2Rᵣₑₗₐₜᵢᵥₑ) is at least 0.5.

In a correspondingly preferred aspect of the invention, (GLP-2Rᵣₑₗₐₜᵢᵥₑ)>(GLP-1Rᵣₑₗₐₜᵢᵥₑ). In a more preferred aspect of the invention, (GLP-2Rᵣₑₗₐₜᵢᵥₑ)>2 (GLP-1Rᵣₑₗₐₜᵢᵥₑ). In an even more preferred aspect of the invention, (GLP-2Rᵣₑₗₐₜᵢᵥₑ)>10 (GLP-1Rᵣₑₗₐₜᵢᵥₑ).

The invention further relates to peptides comprising a peptide sequence according to SEQ ID NO: 1 or a lipidated peptide analogs hereof having no more than 2 deviations from the amino acid sequence of SEQ ID NO: 1;

(SEQ ID NO: 1)
H-X₂-D-G-X₅-F-X₇-X₈-X₉-X₁₀-S-X₁₂-Y-X₁₄-X₁₅-X₁₆-L-A-X₁₉-
X₂₀-X₂₁-F-I-X₂₄-W-L-X₂₇-X₂₈-X₂₉-X₃₀-X₃₁-X₃₂-X₃₃,

wherein
X₂ is Gly, Ala, Aib (2-aminoisobutyric acid), Sar (N-methyl glycin);
X₅ is Thr, Ser;
X₇ is Thr, Ser;
X₈ is Thr, Asp, Ser, Glu;
X₉ is Asp, Glu;
X₁₀ is Leu, Nle (Norleucine), Met, Val, Tyr;
X₁₂ is Thr, Ser, Ala;
X₁₄ is Leu, Nle (Norleucine), Met, Val;
X₁₅ is Asp, Glu;
X₁₆ is Ala, Asn, Gln, Gly, Ser, Glu, Asp, Arg, Lys;
X₁₉ is Ala, Val, Leu;
X₂₀ is Arg, Lys, His;
X₂₁ is Asp, Glu;
X₂₄ is Ala, Asn, Asp, Gln, Glu, Lys, Arg;
X₂₇ is Ile, Leu, Val, Lys, Arg, Nle (Norleucine);
X₂₈ is Gln, Asn, Lys, Arg;
X₂₉ is Thr, Ser, Lys, Arg;
X₃₀ is Lys, Arg;
X₃₁ is Ile or absent;
X₃₂ is Thr or absent;
X₃₃ is Asp or absent,
   or a pharmaceutically acceptable salt or solvate thereof.

In a preferred aspect of the invention, the peptide comprises, at the N-terminal residue, a R¹ group being hydrogen, methyl, acetyl, formyl, benzoyl or trifluoroacetyl, and at the C-terminal residue, an R² group being NH₂ or OH.

In a preferred aspect of the invention, X₇ is Thr, X₉ is Glu and X₁₉ is Ala. Thus, in a preferred aspect of the invention the peptide comprises or consists of a peptide sequence according to SEQ ID NO: 2 or a lipidated peptide analog thereof

R¹-H-G-D-G-S-F-T-X₈-E-X₁₀-S-T-Y-L-D-X₁₆-L-A-A-R-D-F-I-X₂₄-W-L-I-Q-T-K-X₃₁-X₃₂-X₃₃-R² (SEQ ID NO: 2), wherein
X₅ is Thr, Asp, Ser, Glu;
X₁₀ is Leu, Nle (Norleucine), Met, Val, Tyr;
X₁₆ is Ala, Asn, Gln, Gly, Ser, Glu, Asp, Arg, Lys;
X₂₄ is Ala, Asn, Asp, Gln, Glu, Lys, Arg;
X₃₁ is Ile or absent;
X₃₂ is Thr or absent;
X₃₃ is Asp or absent;
   wherein R¹ is hydrogen, methyl, acetyl, formyl, benzoyl, trifluoroacetyl, and R² is NH₂ or OH.

In a preferred aspect of the invention, X₈ is Asp or Ser. In an even more preferred aspect, X₈ is Ser.

In a preferred aspect of the invention, X₁₀ is Leu or Nle (Norleucine).

In a preferred aspect of the invention, X₁₆ is Ala or Asn.

In a preferred aspect of the invention, X₂₄ is Ala or Asn.

In a preferred aspect of the invention, X₃₁, X₃₂, and X₃₃ are Ile, Thr, and Asp.

In another preferred aspect of the invention, X₃₁, X₃₂, and X₃₃ are absent.

In one of the most preferred aspects of the invention, the peptides are selected among

(SEQ ID NO: 3)
R¹-H-G-D-G-S-F-T-D-E-L-S-T-Y-L-D-A-L-A-A-R-D-F-I-A-
W-L-I-Q-T-K-R²;
(SEQ ID NO: 4)
R¹-H-G-D-G-S-F-T-S-E-L-S-T-Y-L-D-A-L-A-A-R-D-F-I-N-
W-L-I-Q-T-K-R²;
(SEQ ID NO: 5)
R¹-H-G-D-G-S-F-T-D-E-L-S-T-Y-L-D-N-L-A-A-R-D-F-I-A-
W-L-I-Q-T-K-I-T-D-R²;
(SEQ ID NO: 6)
R¹-H-G-D-G-S-F-T-S-E-L-S-T-Y-L-D-A-L-A-A-R-D-F-I-A-
W-L-I-Q-T-K-I-T-D-R²;
and
(SEQ ID NO: 7)
R¹-H-G-D-G-S-F-T-S-E-L-S-T-Y-L-D-N-L-A-A-R-D-F-I-N-
W-L-I-Q-T-K-I-T-D-R²,
(SEQ ID NO: 8)
R¹-H-G-D-G-S-F-T-S-E-L-S-T-Y-L-D-N-L-A-A-R-D-F-I-A-
W-L-I-Q-T-K-R²;
(SEQ ID NO: 9)
R¹-H-G-D-G-S-F-T-S-E-L-S-T-Y-L-D-A-L-A-A-R-D-F-I-A-
W-L-I-Q-T-K-R²;
(SEQ ID NO: 10)
R¹-H-G-D-G-S-F-T-S-E-L-S-T-Y-L-D-N-L-A-A-R-D-F-I-A-
W-L-I-Q-T-K-I-T-D-R²;
(SEQ ID NO: 11)
R¹-H-G-D-G-S-F-T-D-E-L-S-T-Y-L-D-A-L-A-A-R-D-F-I-A-
W-L-I-Q-T-K-I-T-D-R²;
(SEQ ID NO: 12)
R¹-H-G-D-G-S-F-T-D-E-L-S-T-Y-L-D-N-L-A-A-R-D-F-I-N-
W-L-I-Q-T-K-R²;
(SEQ ID NO: 13)
R¹-H-G-D-G-S-F-T-D-E-L-S-T-Y-L-D-A-L-A-A-R-D-F-I-N-
W-L-I-Q-T-K-I-T-D-R²;
(SEQ ID NO: 14)
R¹-H-G-D-G-S-F-T-S-E-L-S-T-Y-L-D-A-L-A-A-R-D-F-I-N-
W-L-I-Q-T-K-I-T-D-R²;
(SEQ ID NO: 15)
R¹-H-G-D-G-S-F-T-D-E-[Nle]-S-T-Y-L-D-N-L-A-A-R-D-F-
I-A-W-L-I-Q-T-K-R²;
(SEQ ID NO: 16)
R¹-H-G-D-G-S-F-T-D-E-[Nle]-S-T-Y-L-D-N-L-A-A-R-D-F-
I-A-W-L-I-Q-T-K-I-T-D-R²;
(SEQ ID NO: 17)
R¹-H-G-D-G-S-F-T-S-E-[Nle]-S-T-Y-L-D-N-L-A-A-R-D-F-
I-A-W-L-I-Q-T-K-I-T-D-R²;
(SEQ ID NO: 18)
R¹-H-G-D-G-S-F-T-S-E-[Nle]-S-T-Y-L-D-A-L-A-A-R-D-F-
I-A-W-L-I-Q-T-K-I-T-D-R²;

In a more preferred aspect of the invention, the peptides are selected among the peptides of SEQ ID NO: 3-7.

In a more preferred aspect of the invention, the peptides are peptides or lipidated analogs of the peptide of SEQ ID NO: 3.

In a more preferred aspect of the invention, the peptides are peptides or lipidated analogs of the peptide of SEQ ID NO: 6.

In highly preferred embodiments of the invention, the peptides comprise one or more, preferably only one, serum albumin binding side chain(s). Preferably, the serum albumin binding side chain is attached to the side chain of an amino acid residue, such as the side chain of lysine (i.e. ε-N-alkylated lysine), 2,3-diaminopropionic acid, 2,4-diaminobutyric acid, ornithine, O-aminopropylserine or longer O-alkylated serines containing a primary amino group.

Preferably, the serum albumin binding amino acid residue is inserted as a substitution into the sequence of SEQ ID NO: 1, whereby the amino acid sequence of the sequence of SEQ ID NO: 1 is (possibly) altered (understood according to the present invention as a “deviation” from the sequence of SEQ ID NO: 1). The introduced deviations may influence the properties of the peptides in terms of GLP-1 and GLP-2 receptor activity, without, however, deviating the resulting serum albumin binding peptides from the scope of the present invention. The skilled person understands that the introduction of one or more serum albumin binding side chains offer the possibility of an extended half-life in vivo in return of a (possible) reduction in GLP-1 and/or GLP-2 receptor activity.

The serum albumin binding amino acid residue comprises a serum albumin binding side chain. Preferably, serum albumin binding side chains according to the present invention are side chains comprising a lipid molecule. In broad terms, the amino acid residues comprising a serum albumin binding side chain according to the present invention are lipidated amino acid residues.

In one embodiment, the serum albumin binding side chain comprises an alkyl chain with at least 14 carbon atoms, wherein said alkyl chain comprises a distal carboxylic acid or a distal tetrazole group and wherein said alkyl chain comprises a proximal carbonyl group.

In one embodiment, the serum albumin binding side chain comprises the structure A, wherein A is selected among

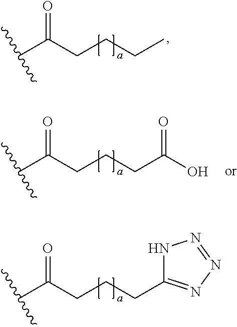

where a is at least 10.

In a preferred embodiment of the present invention, the serum albumin binding side chain is selected from the group consisting of A-B-C-, A-B- and A-C-, wherein A is

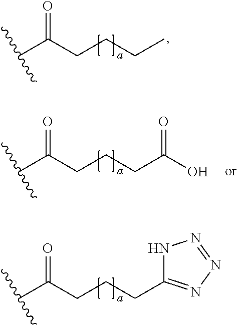

where a is selected from the group consisting of 10, 11, 12, 14, 15, 16, 17 and 18 and B is selected from

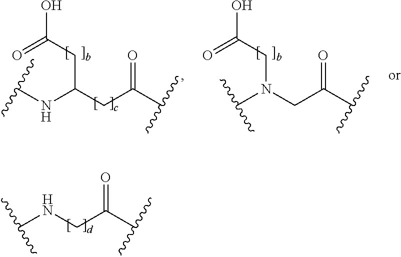

wherein b is selected from the group consisting of 0, 1, and 2, c is selected from the group consisting of 0, 1 and 2, and d is selected from the group consisting of 1, 2, 3, 4, 5, 6, 7, 8, 9, 10, 11 and 12.

C is a spacer molecule, such as at least one D, E, S, T, K, R, G, A, Ornithine, Citrulline, 8-amino-3,6-dioxaoctanoic acid or 12-amino-4,7,10-trioxaoctadecanoic acid molecule.

Serum albumin binding side chains according to the present invention are described e.g. in WO2011058165.

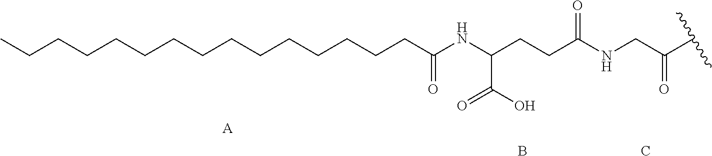

An example of A-B- is:

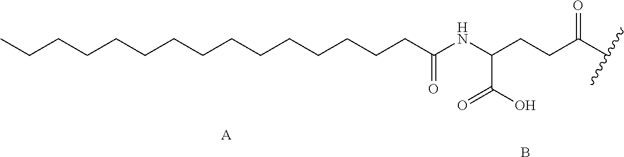

An example of A-C- is:

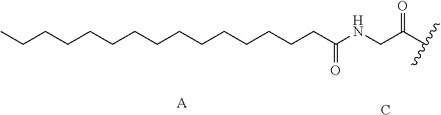

In one embodiment, the serum albumin binding side chain comprises at least one dicarboxylic acid, such as hexadecanedioic acid, octadecanedioic acid or dodecanedioic acid.

In a highly preferred embodiment, the serum albumin binding side chain consists of the structure A-B, attached to a lysine residue.

A highly preferred example of such embodiment is show below, where A is

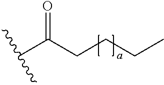

### and B is

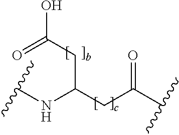

attached to a lysine residue.

A highly preferred amino acid side chain (termed K(C16-yE-) is shown below:

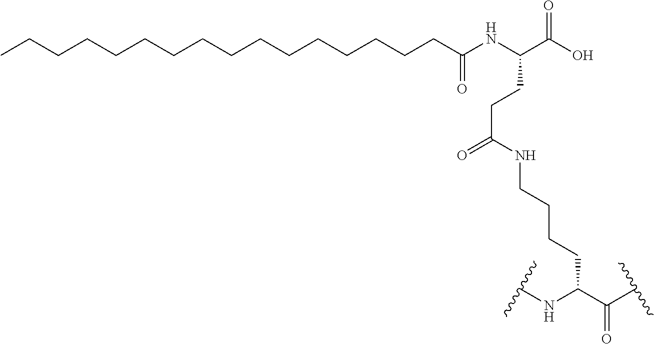 [SMILES: *N[C@H](CCCCNC(=O)CC[C@H](NC(=O)CCCCCCCCCCCCCCCC)C(=O)O)C(*)=O]

It is believed that the negative charge in the distal end of the dicarboxylic acid increases affinity of the peptides to serum albumin. In one embodiment, the fatty di-acid or tetrazole may be attached to a spacer, such as a negatively charged amino acid, e.g., L-gamma-glutamate. In one embodiment, the alkyl chain, optionally comprising a dicarboxylic acid or a tetrazole group, may be attached to a spacer. In one embodiment, the combined alkyl chain, optionally comprising a dicarboxylic acid or a tetrazole group, and the negatively charged amino acid may be separated with a spacer.

In a preferred aspect, the invention comprises a lipidated peptide analog of the above listed SEQ ID NO: 1-18, said lipidated peptides comprising at least one lipidated amino acid residue. Preferably, the lipidated peptides comprise only one lipidated amino acid residue in order to minimise the impact of the lipidation on the receptor activity of the peptides.

As seen in the examples, based on the peptide with the SEQ ID NO: 6, it was shown that introduction of a serum albumin binding side chain at positions X₇, X₈, X₁₁, X₁₂, X₁₄, X₁₆, X₁₇, X₂₀ provides functional peptides.

Accordingly, in a preferred embodiment of the invention, the peptides are lipidated analogs of the peptide of SEQ ID NO: 1, comprising a lipidation introduced on an amino acid residue at a position selected among positions X₇, X₈, X₁₁, X₁₂, X₁₄, X₁₆, X₁₇ and X₂₀.

In a more preferred embodiment of the invention, the peptides are lipidated analogs of the peptide of SEQ ID NO: 1, comprising a lipidation introduced by substituting an amino acid residue with a lipidated serum albumin binding amino acid residue at a position selected among positions X₁₁, X₁₂, X₁₄, X₁₆, X₁₇ and X₂₀.

In a more preferred embodiment of the invention, the peptides are lipidated analogs of the peptide of SEQ ID NO: 1, comprising a lipidation introduced on an amino acid residue at a position selected among positions X₁₂, X₁₄, X₁₆ and X₁₇.

In a more preferred embodiment of the invention, wherein the peptides are lipidated analogs of the peptide of SEQ ID NO: 1, the lipidation is introduced on the amino acid residue at positions X₁₄ or X₁₇.

In a highly preferred embodiment of the invention, the peptides are lipidated analogs of the peptide of SEQ ID NO: 6, comprising a lipidation introduced on an amino acid residue at a position selected among positions X₇, X₈, X₁₁, X₁₂, X₁₄, X₁₆, X₁₇ and X₂₀.

In a highly preferred embodiment of the invention, the peptides are lipidated analogs of the peptide of SEQ ID NO: 6, comprising a lipidation introduced on an amino acid residue at a position selected among positions X₁₁, X₁₂, X₁₄, X₁₆, X₁₇ and X₂₀.

In a highly preferred embodiment of the invention the peptides are lipidated analogs of the peptide of SEQ ID NO: 6, comprising a lipidation introduced on an amino acid residue at a position selected among positions X₁₂, X₁₄, X₁₆ and X₁₇.

In a highly preferred embodiment of the invention, wherein the peptides are lipidated analogs of the peptide of SEQ ID NO: 6, the lipidation is introduced on the amino acid residue at positions X₁₄ or X₁₇.

Particularly, highly preferred lipidated analogs of the peptide of SEQ ID NO: 6, are selected among the following sequence ID NOs:

(SEQ ID NO: 19)
R¹-H-G-D-G-S-F-[K(C16-yE-)]-S-E-L-S-T-Y-L-D-A-L-A-
A-R-D-F-I-A-W-L-I-Q-T-K-I-T-D-R²;
(SEQ ID NO: 20)
R¹-H-G-D-G-S-F-T-[K(C16-yE-)]-E-L-S-T-Y-L-D-A-L-A-
A-R-D-F-I-A-W-L-I-Q-T-K-I-T-D-R²;
(SEQ ID NO: 21)
R¹-H-G-D-G-S-F-T-S-E-L-[K(C16-yE-)]-T-Y-L-D-A-L-A-
A-R-D-F-I-A-W-L-I-Q-T-K-I-T-D-R²;
(SEQ ID NO: 22)
R¹-H-G-D-G-S-F-T-S-E-L-S-[K(C16-yE-)]-Y-L-D-A-L-A-
A-R-D-F-I-A-W-L-I-Q-T-K-I-T-D-R²;
(SEQ ID NO: 23)
R¹-H-G-D-G-S-F-T-S-E-L-S-T-Y-[K(C16-yE-)]-D-A-L-A-
A-R-D-F-I-A-W-L-I-Q-T-K-I-T-D-R²;
(SEQ ID NO: 24)
R¹-H-G-D-G-S-F-T-S-E-L-S-T-Y-L-D-[K(C16-yE-)]-L-A-
A-R-D-F-I-A-W-L-I-Q-T-K-I-T-D-R²;
(SEQ ID NO: 25)
R¹-H-G-D-G-S-F-T-S-E-L-S-T-Y-L-D-A-[K(C16-yE-)]-A-
A-R-D-F-I-A-W-L-I-Q-T-K-I-T-D-R²;
and
(SEQ ID NO: 26)
R¹-H-G-D-G-S-F-T-S-E-L-S-T-Y-L-D-A-L-A-A-
[K(C16-yE-)]-D-F-I-A-W-L-I-Q-T-K-I-T-D-R².

One particularly highly preferred lipidated analog of the peptide of SEQ ID NO: 6 is the peptide having the sequence ID NO 23.

Another particularly highly preferred lipidated analog of the peptide of SEQ ID NO: 6 is the peptide having the sequence ID NO 25.

As seen in the experimental part below, the dual compositions or peptides according to the invention have surprisingly been observed to provide beneficial pharmacological effects. Thus, the invention further relates to the use of the above dual peptides according to the invention for treatment or prophylactic treatment of a human or animal subject.

In particular, the invention relates to the use of the above dual compositions or peptides according to the invention for treatment or prophylactic treatment of gut and brain related diseases of a human or animal subject. Further, the invention relates to the use of the above dual peptides according to the invention for the treatment or prophylactic treatment of diseases associated with both GLP-1 and GLP-2 in a human or animal subject.

In particular, the invention relates to the use of the above dual GLP1R-GLP2R agonist compositions or peptides according to the invention for treatment or prophylactic treatment of gastrointestinal disorders as well as stomach and intestinal-related disorders in a human or animal subject. Gastrointestinal disorders include the disorders of the upper gastrointestinal tract of the oesophagus. Stomach and intestinal-related disorders include ulcers of any aetiology (e.g. peptic ulcers, Zollinger-Ellison syndrome, drug-induced ulcers, ulcers related to infections or other pathogens), digestion disorders, malabsorptions, short bowel syndrome, cul-de-sac syndrome, inflammatory bowel diseases (Crohn's disease and ulcerative colitis), celiac sprue, hypogammaglobulinemic sprue, and chemotherapy and/or radiation therapy-induced mucositis and diarrhea.

Moreover, the above dual compositions or GLP1R-GLP2R agonist peptides according to the invention are also useful for preventing or treating metabolic syndrome, obesity, diabetes, non-alcoholic steatohepatitis (NASH) and preventing or treating inflammation in metabolically important tissues including, liver, fat, pancreas, kidney, gut. Especially, the peptides according to the invention are believed to be effective in the treatment of non-alcoholic steatohepatitis (NASH).

Further, accelerated synthesis of glucose, the primary fuel of tissue repair, is an important metabolic change after surgery, and occurs at the expense of body protein and energy stores (Gump et al. 1974, J. Trauma 14: 378-88; Black et al. 1982, Ann. Surg. 196: 420-35). These changes have previously been attributed to the gluco-regulatory stress hormones and other catabolic factors, such as cytokines, that are released as a response to trauma. The more marked the change toward catabolism, the greater is the morbidity, and the slower is the recovery of the patient (Thorell et al. 1993, Eur. J. Surg. 159: 593-99; Chernow et al. 1987, Arch. Intern. Med. 147: 1273-8). Thus, the above dual compositions and GLP1R-GLP2R agonist peptides according to the invention are also useful for preventing or treating surgical trauma by improving recovery after surgery by preventing the catabolic reaction and insulin resistance caused by surgical trauma.

Particularly, the compositions and peptides according to the invention are suitable for use in the treatment or prophylactic treatment of a human or animal subject, said treatment or prophylactic treatment being the treatment or prophylactic treatment of a condition related to short bowel syndrome.

Accordingly, the present invention further relates to a pharmaceutical or veterinary composition comprising a peptide according to the invention and at least one pharmaceutical or veterinary excipient.

The dual analogs of the present invention, or salts or derivatives thereof, may be formulated as pharmaceutical compositions prepared for storage or administration, and which comprise a therapeutically effective amount of a peptide of the present invention, or a salt or derivative thereof, in a pharmaceutically acceptable carrier.

The skilled person is familiar with the provision of suitable pharmaceutical formulations or compositions comprising peptide pharmaceuticals.

The invention further relates to the use of a peptide or composition according to the invention for medical or veterinary treatment of a human or animal subject.

Preferably, the pharmaceutical or veterinary compositions according to the invention are in a unit dosage form.

The invention also relates to a method of treatment of a human or animal subject, the method comprising administering a peptide or composition according to the invention to a human or animal subject.

Method of Production:

It is preferred to synthesize the analogs of the invention by means of solid-phase or liquid-phase peptide synthesis. Such synthesis is well-known to the skilled person.

However, the dual analogs may be synthesized in a number of ways including, for example, a method comprising:

(a) synthesizing the peptide by means of solid-phase or liquid-phase peptide synthesis and recovering the synthetic peptide thus obtained; or
(b) when the peptide consists of naturally occurring amino acids, expressing a nucleic acid construct that encodes the peptide in a host cell and recovering the expression product from the host cell culture; or
(c) when the peptide consists of naturally occurring amino acids, effecting cell-free in vitro expression of a nucleic acid construct that encodes the peptide and recovering the expression product; or a combination of methods of (a), (b), and (c) to obtain fragments of the peptide, subsequently joining (e.g., ligating) the fragments to obtain the peptide, and recovering the peptide.

Thus, the invention further relates to a nucleic acid molecule comprising a nucleic acid sequence encoding the peptide of the invention.

The invention further relates to a method of producing a peptide of the invention, the method comprising a step of providing expression of the nucleic acid molecule of above and purifying the product thus produced.

### EXAMPLES

### Example 1

### Peptide Synthesis and Purification

All peptides were synthesized by solid-phase peptide synthesis using 9-fluorenylmethyloxycarbonyl (Fmoc) as N-α-amino protecting group and suitable common protection groups for side-chain functionalities. The syntheses were performed on MultiSynTech Syroll (Biotage) using TentaGel S Ram resin (0.1 mmol scale; loading 0.24 mmol/g). The couplings were performed using N,N′-diisopropylcarbodiimide (DIC) as coupling reagent and Oxyma Pure as additive in N,N-dimethylformaide (DMF). The couplings were performed for 2×2 hours at room temperature. After each coupling, the resin was washed with N-methylpyrrolidone (NMP) (2×2.5 ml), dichloromethane (2.5 ml) and NMP (2×2.5 ml). For N-α-deprotection, piperidine in NMP (40%) was added to the resin for initial deprotection (3 min) followed by second deprotection using piperidine in NMP (20%) for 17 min. The resin was washed with NMP (2×2.5 ml) dichloromethane (2.5 ml) and NMP (2×2.5 ml). The lipidations were site-selectively attached on a ε amine of Lys. The Lys was side chain protected with Alloc. The final N-terminally located amino acid (His) was N protected with Boc. The Alloc group was removed using tetrakis(triphenylphosphine)palladium(0) and dimethylamine borane complex in degassed dichloromethane for 2 hours at room temperature. After washing extensively with DCM, 20% piperidine in NMP and NMP, the lipidation was performed using manual synthesis. The couplings were performed using HATU (N-[(dimethylamino)-1H-1,2,3-triazolo[4,5-b]pyridin-1-ylmethylene]-N-methylmethanaminium hexafluorophosphate N-oxide) as coupling reagent and DIEA as base and Fmoc removals were performed using 20% piperidine in NMP.

For the peptide cleavage from the solid-support and amino acid side-chain deprotections, the resins were washed using dichloromethane and dried at room temperature for 30 minutes. The resins were treated with trifluoroacetic acid (TFA)/triethylsilane (TES)/water (95/2.5/2.5; 2 hours; room temperature). The TFA-peptide mixtures were collected and diethylether was added leading to precipitation of the crude peptides. Ether was removed by centrifugation and decantation. The peptides were purified by preparative RP-HPLC using Waters 150 LC system equipped with a Gemini-NX, AXIA packed, 5 μm C-18, 110 Å, 30-100 mm column and a fraction collector using a 5-60% gradient of buffer B (0.1% TFA in acetonitrile) in buffer A (0.1% TFA in water) over 35 min at 30 ml/min. Purified peptides were characterized by UPLC-ESI-MS (Waters) equipped with a Acquity UPLC BEH C-18 1.7 μm, 2.1-100 mm using a 0-100% gradient of buffer B (5% water, 0.1% formic acid in acetonitrile) in buffer A (5% acetonitrile, 0.1% formic acid in water) over 6 min. All peptides were determined by UV to be more than 95% pure.

The compounds below were synthesised using the above techniques.

(SEQ ID NO: 3)
R¹-H-G-D-G-S-F-T-D-E-L-S-T-Y-L-D-A-L-A-A-R-D-F-I-
A-W-L-I-Q-T-K-R²;
(SEQ ID NO: 4)
R¹-H-G-D-G-S-F-T-S-E-L-S-T-Y-L-D-A-L-A-A-R-D-F-I-
N-W-L-I-Q-T-K-R²;
(SEQ ID NO: 5)
R¹-H-G-D-G-S-F-T-D-E-L-S-T-Y-L-D-N-L-A-A-R-D-F-I-
A-W-L-I-Q-T-K-I-T-D-R²;
(SEQ ID NO: 6)
R¹-H-G-D-G-S-F-T-S-E-L-S-T-Y-L-D-A-L-A-A-R-D-F-I-
A-W-L-I-Q-T-K-I-T-D-R²;
(SEQ ID NO: 7)
R¹-H-G-D-G-S-F-T-S-E-L-S-T-Y-L-D-N-L-A-A-R-D-F-I-
N-W-L-I-Q-T-K-I-T-D-R²;
(SEQ ID NO: 8)
R¹-H-G-D-G-S-F-T-S-E-L-S-T-Y-L-D-N-L-A-A-R-D-F-I-
A-W-L-I-Q-T-K-R²;
(SEQ ID NO: 9)
R¹-H-G-D-G-S-F-T-S-E-L-S-T-Y-L-D-A-L-A-A-R-D-F-I-
A-W-L-I-Q-T-K-R²;
(SEQ ID NO: 10)
R¹-H-G-D-G-S-F-T-S-E-L-S-T-Y-L-D-N-L-A-A-R-D-F-I-
A-W-L-I-Q-T-K-I-T-D-R²;
(SEQ ID NO: 11)
R¹-H-G-D-G-S-F-T-D-E-L-S-T-Y-L-D-A-L-A-A-R-D-F-I-
A-W-L-I-Q-T-K-I-T-D-R²;
(SEQ ID NO: 12)
R¹-H-G-D-G-S-F-T-D-E-L-S-T-Y-L-D-N-L-A-A-R-D-F-I-
N-W-L-I-Q-T-K-R²;
(SEQ ID NO: 13)
R¹-H-G-D-G-S-F-T-D-E-L-S-T-Y-L-D-A-L-A-A-R-D-F-I-
N-W-L-I-Q-T-K-I-T-D-R²;
(SEQ ID NO: 14)
R¹-H-G-D-G-S-F-T-S-E-L-S-T-Y-L-D-A-L-A-A-R-D-F-I-
N-W-L-I-Q-T-K-I-T-D-R²;
(SEQ ID NO: 15)
R¹-H-G-D-G-S-F-T-D-E-[Nle]-S-T-Y-L-D-N-L-A-A-R-D-
F-I-A-W-L-I-Q-T-K-R²;
(SEQ ID NO: 16)
R¹-H-G-D-G-S-F-T-D-E-[Nle]-S-T-Y-L-D-N-L-A-A-R-D-
F-I-A-W-L-I-Q-T-K-I-T-D-R²;
(SEQ ID NO: 17)
R¹-H-G-D-G-S-F-T-S-E-[Nle]-S-T-Y-L-D-N-L-A-A-R-D-
F-I-A-W-L-I-Q-T-K-I-T-D-R²;
(SEQ ID NO: 18)
R¹-H-G-D-G-S-F-T-S-E-[Nle]-S-T-Y-L-D-A-L-A-A-R-D-
F-I-A-W-L-I-Q-T-K-I-T-D-R²;
(SEQ ID NO: 19)
R¹-H-G-D-G-S-F-[K(C16-yE-)]-S-E-L-S-T-Y-L-D-A-L-A-
A-R-D-F-I-A-W-L-I-Q-T-K-I-T-D-R²;
(SEQ ID NO: 20)
R¹-H-G-D-G-S-F-T-[K(C16-yE-)]-E-L-S-T-Y-L-D-A-L-A-
A-R-D-F-I-A-W-L-I-Q-T-K-I-T-D-R²;
(SEQ ID NO: 21)
R¹-H-G-D-G-S-F-T-S-E-L-[K(C16-yE-)]-T-Y-L-D-A-L-A-
A-R-D-F-I-A-W-L-I-Q-T-K-I-T-D-R²;
(SEQ ID NO: 22)
R¹-H-G-D-G-S-F-T-S-E-L-S-[K(C16-yE-)]-Y-L-D-A-L-A-
A-R-D-F-I-A-W-L-I-Q-T-K-I-T-D-R²;
(SEQ ID NO: 23)
R¹-H-G-D-G-S-F-T-S-E-L-S-T-Y-[K(C16-yE-)]-D-A-L-A-
A-R-D-F-I-A-W-L-I-Q-T-K-I-T-D-R²;
(SEQ ID NO: 24)
R¹-H-G-D-G-S-F-T-S-E-L-S-T-Y-L-D-[K(C16-yE-)]-L-A-
A-R-D-F-I-A-W-L-I-Q-T-K-I-T-D-R²;
(SEQ ID NO: 25)
R¹-H-G-D-G-S-F-T-S-E-L-S-T-Y-L-D-A-[K(C16-yE-)]-A-
A-R-D-F-I-A-W-L-I-Q-T-K-I-T-D-R²;
and
(SEQ ID NO: 26)
R¹-H-G-D-G-S-F-T-S-E-L-S-T-Y-L-D-A-L-A-A-
[K(C16-yE-)]-D-F-I-A-W-L-I-Q-T-K-I-T-D-R².

### Example 2

### GLP1 and GLP2 Receptor Efficacy Assays

In vitro functional screens were performed either at Euroscreen (Belgium) or at DiscoveRx (USA). Dose response curves were performed in parallel with native human GLP-1 and GLP-2 as reference compounds. Exemplified results are listed in Table 1 and 2.

TABLE 1
Receptor potency

| Compound | GLP-1R, EC₅₀ (nM) | GLP-2R, EC₅₀ (nM) |
|---|---|---|
| GLP-1 | 0.026 | >1000 |
| GLP-2 | ND | 0.45 |
| SEQ ID NO 3 | 0.28 | 0.19 |
| SEQ ID NO 4 | 0.04 | 1.38 |
| SEQ ID NO 5 | 2.02 | 0.29 |
| SEQ ID NO 6 | 0.16 | 0.07 |
| SEQ ID NO 7 | 0.18 | 2.28 |
| SEQ ID NO 10 | 0.16 | 6.53 |
| SEQ ID NO 11 | 3.99 | 0.33 |
| SEQ ID NO 13 | 4.16 | 0.14 |
| SEQ ID NO 15 | 0.40 | 7.93 |
| SEQ ID NO 16 | 1.79 | 1.52 |
| SEQ ID NO 17 | 0.08 | 7.17 |
| SEQ ID NO 18 | 0.09 | 7.06 |

TABLE 2
Receptor potency

| Compound | GLP-1R, EC₅₀ (nM) | GLP-2R, EC₅₀ (nM) |
|---|---|---|
| GLP-1 | 0.004 | >100 |
| GLP-2 | nd | 0.0227 |
| SEQ ID NO 19 | >10 | >100 |
| SEQ ID NO 20 | >10 | 9.445 |
| SEQ ID NO 21 | >10 | 4.248 |
| SEQ ID NO 22 | 0.543 | 6.610 |
| SEQ ID NO 23 | 0.169 | 1.057 |
| SEQ ID NO 24 | 0.338 | 5.597 |
| SEQ ID NO 25 | 0.319 | 1.928 |
| SEQ ID NO 26 | 0.658 | >100 |

### Example 3

### Effect on Gut Proliferation in C57BL/6J Mice

PoC Study 1—Using Native GLP-1 and GLP-2.

Forty (40) female C57BL/6J mice were purchased from Janvier (France). Based on the body weight recorded on day −1, mice were randomized into the following four study groups (n=10 per group): Group 1: Vehicle, BID, SC. Group 2: native GLP-1 (GLP-1(7-36)-amide) 3.0 mg/kg, BID, SC. Group 3: native GLP-2 3.0 mg/kg, BID, SC. Group 4: native GLP-1 3.0 mg/kg+native GLP-2 3.0 mg/kg, BID, SC. Dosing volume was 5 ml/kg, SC. Compounds were dissolved in PBS buffer containing 3% mannitol and 0.6% L-His (pH 9.0). The subcutaneous injections were placed at the lower back. On study day 8, the mice were terminated and the GI tract was removed for weight determination. Results on gut wet weight are listed below in Table 3.

TABLE 3
Total intestine weight - PoC 1
Day 8
(mg ± S.E.M.)

| Dose | 0 mg/kg | 3 mg/kg | 3 + 3 mg/kg |
|---|---|---|---|
| Vehicle | 1009 ± 33.5 | | |
| GLP-1 | | 1090 ± 34.6 | |
| GLP-2 | | 1256 ± 30.1 *** | |
| GLP-1 + GLP-2 | | | 1354 ± 21.5 ***, ^{#} |

Mean ± SEM (n = 9-10);
*** P < 0.001 compared to vehicle; (One-way ANOVA w/Dunnett's post hoc test)
^{#} P < 0.05 compared to GLP-2; (Student's t-test)

### Results:

The present data show that only seven days of dosing with native GLP-2 is able to significantly increase intestinal weight by approx. 25% compared to vehicle treatment. Surprisingly, the co-administration of native GLP-2 together with native GLP-1 lead to an even further increase in intestinal weight of approx. 34% (Table 3). The additional increase in intestinal weight following co-treatment was statistically significant compared to native GLP-2 treatment alone (P=0.015, student's t-test).

### Example 4

### Effect on Acute Food Intake in C57BL/6J Mice

The C57BL/6J mice arrived 7 days prior to the initiation of the study. During these days, animals were handled daily to accustom them to the experimental paradigm. At Day −1, the mice were randomised according to body weight to participate in one of following drug treatment groups (n=6-8 in each group): Group 1: vehicle. Group 2: liraglutide 0.2 mg/kg. Group 3-5: SEQ ID NO 3; 3.0; 1.0; 0.3 mg/kg. Group 6-8: SEQ ID NO 4; 3.0; 1.0; 0.3 mg/kg. Group 9-11: SEQ ID NO 5; 3.0; 1.0; 0.3 mg/kg. Group 12-14: SEQ ID NO 6; 3.0; 1.0; 0.3 mg/kg. Group 15-17: SEQ ID NO 7; 3.0; 1.0; 0.3 mg/kg. Three days prior to initiation of dosing, all animals were handled daily and accustomed to the experimental conditions by subcutaneous mock injections (i.e. nipping the skin of the back). Baseline food intake was monitored four days prior to the study. Mice were randomized according to body weight on Day −1. Animals were subjected to two separate injections. First acute dose was administered prior to lights out at Day 0 and food intake was monitored 24 hours post dosing. On Day 3, mice were re-randomised into new treatment groups and dosed prior to lights out. Food intake was monitored 24 hours post dosing. Body weight was measured daily from Day −5 and throughout the study. The study was terminated on Day 5. The analogs were dosed in 3.0; 1.0; and 0.3 mg/kg doses SC and liraglutide in 0.2 mg/kg. Dosing took place prior to lights out in the afternoon (between 13.30 and 14.00). Doses were administered subcutaneously (dose volume 5 ml/kg). Food intake data was collected using the HM-2 system, an online computerized feeding system using digital weighing cells. Food intake and body weight were measured from Day −7 and throughout the study. The vehicle was prepared by dissolving 3% mannitol and 0.6% L-His in phosphate buffer saline and adjusting the pH to 9.0. The results are listed below in Table 4.

TABLE 4
Total food intake (g/animal)
Day 0: 0-4.5 hours (lights off)

| Dose | 0 mg/kg | 0.2 mg/kg | 3 mg/kg | 1 mg/kg | 0.3 mg/kg |
|---|---|---|---|---|---|
| Vehicle | 1.45 ± 0.50 | | | | |
| Liraglutide | | 0.08 ± 0.03 *** | | | |
| SEQ ID NO 3 | | | 0.30 ± 0.08 ** | 0.66 ± 0.15 | 0.76 ± 0.37 |
| SEQ ID NO 4 | | | 0.25 ± 0.08 ** | 0.79 ± 0.15 | 0.93 ± 0.06 |
| SEQ ID NO 5 | | | 0.26 ± 0.08 ** | 0.83 ± 0.18 | 0.69 ± 0.19 |
| SEQ ID NO 6 | | | 0.06 ± 0.03 *** | 0.13 ± 0.08 *** | 0.33 ± 0.17 ** |
| SEQ ID NO 7 | | | 0.28 ± 0.12 ** | 0.47 ± 0.08 * | 1.05 ± 0.41 |

Mean ± SEM (n = 8-10);
* P < 0.05,
** P < 0.01,
*** P < 0.001 compared to vehicle; (One-way ANOVA w/Dunnett's post hoc test)

### Results:

It was observed that the tested SEQ ID NO. 3-7 reduced food intake after 4.5 hours in all tested doses compared to vehicle treated controls. Further, a single dose of SEQ ID NO 6, (0.3-3 mg/kg), a single dose of SEQ ID NO 7 (1-3 mg/kg), and a single dose of SEQ ID NO 3-5 (3 mg/kg) significantly reduced food intake after 4.5 hours compared to vehicle treated controls.

Thus, the results show that the most effective inhibitor of food intake after 4.5 hours is SEQ ID NO 6 (Table 4) which is also the most potent dual agonist and the most potent GLP-2 receptor agonist (Table 1). These results further indicate that dual GLP-1 and GLP-2 receptor activities reduce food intake.

### Example 5

### Effect of GLP-1 and GLP-2 Peptides in Combination on Gut Proliferation in C57BL/6J Mice

### General Protocol Procedure

Forty (40, n=10, 4 groups) male C57BL/6J mice obtained from Taconic, Denmark, were used. At the time of the experiment, the mice had reached an age of 9-10 weeks. The mice were acclimatized for one week in their new environment and offered regular chow diet (Altromin 1324, Brogaarden A/S, Denmark) and domestic quality tap water. Animals were housed two per cage during the study, in a light-, temperature-, and humidity-controlled room (12-hour light: 12-hour dark cycle, lights on/off at 4 AM/4 PM hour; 22±1° C.; 50±10% relative humidity). Mice were randomized into the four study groups based on the body weight recorded on Day −1. Animals were uniquely identified with implantable microchips (Pet ID Microchip, E-vet) in all mice upon arrival. Animals were identified using the WS-2 weight station (MBrose ApS, Faaborg, Denmark) connected to a laptop running the HM02Lab software (Ellegaard Systems, Faaborg, Denmark). The HM02Lab software matches the body weight with ID and calculates the dose directly based on the body weight. All mice were handled for 3 days prior to the experiment to acclimatize the animals to handling and injections.

PoC Study 2—Using Known GLP-1 Analog Liraglutide and GLP-2 Analog Teduglutide

The mice were randomized into three study groups (n=10 per group) based on the body weight recorded on Day −1. The groups were: Group 1: Vehicle, BID, SC. Group 2: liraglutide 0.2 mg/kg, BID, SC. Group 3: teduglutide 1.0 mg/kg, BID, SC. Group 4: liraglutide 0.2 mg/kg and teduglutide 1.0 mg/kg, BID, SC. Dosing volume was 5 ml/kg, SC. Compounds were dissolved in PBS buffer (pH 7.4) containing 3% mannitol and 0.6% L-His to obtain a final concentration of 0.6 mg/ml. Animals were dosed with a 27 g×⅝″ needle (Monoject™, Kendall) connected to a 1 ml syringe (Luer-Lock™, Becton). The subcutaneous injections were placed at the lower back. On study Day 8, the mice were terminated and the GI tract was removed for weight determination. Results on gut wet weight are listed below in Table 5. Results on small intestine weight (Table 6) and large intestine weight (Table 7) are listed below. Results on small intestine length (Table 8) and large intestine length (Table 9) are listed below.

TABLE 5
Gut wet weight - PoC study 2
Day 8
(g ± S.E.M.)

| | |
|---|---|
| Group 1: | 2.2 ± 0.2 ^{###} |
| Vehicle | |
| Group 2: | 3.2 ± 0.3 *** ^{###} |
| Liraglutide, 0.2 mg/kg (BID) | |
| Group 3: | 2.6 ± 0.2 * ^{###} |
| Teduglutide, 1.0 mg/kg (BID) | |
| Group 4: | 4.0 ± 0.4 *** |
| Teduglutide, 1.0 mg/kg + | |
| liraglutide, 0.2 mg/kg (BID) | |

Mean ± SEM (n = 9-10);
* P < 0.05,
** P < 0.01,
*** P < 0.001 compared to vehicle;
^{#} P < 0.05,
^{##} P < 0.01,
^{###} P < 0.001 compared to Group 4; (One-way ANOVA w/Dunnett's post hoc test)

TABLE 6
Small intestine weight - PoC study 2
Day 8
(g ± S.E.M.)

| | |
|---|---|
| Group 1: | 1.1 ± 0.2 ^{###} |
| Vehicle | |
| Group 2: | 1.5 ± 0.3 ** ^{###} |
| Liraglutide, 0.2 mg/kg (BID) | |
| Group 3: | 1.3 ± 0.2 ^{###} |
| Teduglutide, 1.0 mg/kg (BID) | |
| Group 4: | 2.0 ± 0.2 *** |
| Teduglutide, 1.0 mg/kg + | |
| liraglutide, 0.2 mg/kg (BID) | |

Mean ± SEM (n = 9-10);
* P < 0.05,
** P < 0.01,
*** P < 0.001 compared to vehicle;
^{#} P < 0.05,
^{##} P < 0.01,
^{###} P < 0.001 compared to Group 4; (One-way ANOVA w/Dunnett's post hoc test)

TABLE 7
Large intestine weight - PoC study 2 Day 8 (g ± S.E.M.)

| | |
|---|---|
| Group 1: | 0.30 ± 0.04 ^{##} |
| Vehicle | |
| Group 2: | 0.30 ± 0.01 ^{##} |
| Liraglutide, 0.2 mg/kg (BID) | |
| Group 3: | 0.26 ± 0.03 ^{###} |
| Teduglutide, 1.0 mg/kg (BID) | |
| Group 4: | 0.46 ± 0.12 ** |
| Teduglutide, 1.0 mg/kg + | |
| liraglutide, 0.2 mg/kg (BID) | |

Mean ± SEM (n = 9-10);
* P < 0.05,
** P < 0.01,
*** P < 0.001 compared to vehicle;
^{#} P < 0.05,
^{##} P < 0.01,
^{###} P < 0.001 compared to Group 4; (One-way ANOVA w/Dunnett's post hoc test)

TABLE 8
Small intestine length - PoC study 2
Day 8
(cm ± S.E.M.)

| | |
|---|---|
| Group 1: | 20.5 ± 1.6^{###} |
| Vehicle | |
| Group 2: | 24.2 ± 1.5*** |
| Liraglutide, 0.2 mg/kg (BID) | |
| Group 3: | 22.9 ± 1.6**^{#} |
| Teduglutide, 1.0 mg/kg (BID) | |
| Group 4: | 24.8 ± 1.4*** |
| Teduglutide, 1.0 mg/kg + | |
| liraglutide, 0.2 mg/kg (BID) | |

Mean ± SEM (n = 9-10);
*P < 0.05,
**P < 0.01,
***P < 0.001 compared to vehicle;
^{#}P < 0.05,
^{##}P < 0.01,
^{###}P < 0.001 compared to Group 4;
(One-way ANOVA w/ Dunnett's post hoc test)

TABLE 9
Large intestine length - PoC study 2
Day 8
(cm ± S.E.M.)

| | |
|---|---|
| Group 1: | 5.2 ± 0.6^{##} |
| Vehicle | |
| Group 2: | 5.9 ± 0.4 |
| Liraglutide, 0.2 mg/kg (BID) | |
| Group 3: | 5.1 ± 0.6^{##} |
| Teduglutide, 1.0 mg/kg (BID) | |
| Group 4: | 6.1 ± 0.7** |
| Teduglutide, 1.0 mg/kg + | |
| liraglutide, 0.2 mg/kg (BID) | |

Mean ± SEM (n = 9-10);
*P < 0.05,
**P < 0.01,
***P < 0.001 compared to vehicle;
^{#}P < 0.05,
^{##}P < 0.01,
^{###}P < 0.001 compared to Group 4;
(One-way ANOVA w/ Dunnett's post hoc test)

### Results:

The present data show that treatment with GLP-1 analog liraglutide leads to increases in gut wet weight (Table 5), small intestine weight (Table 6), small intestine length (Table 8) and large intestine length (Table 9). It is further shown that treatment with the GLP-2 analog teduglutide leads to increases in gut wet weight (Table 5), small intestine weight (Table 6) and small intestine length (Table 8).

Surprisingly, however, combination treatment with the GLP-1 analog liraglutide and the GLP-2 analog teduglutide is shown to lead to synergistic effects on gut wet weight (Table 5), small intestine weight (Table 6) and large intestine weight (Table 7) as well as on large intestine length (Table 9).

### Example 6

### GLP1R-GLP2R Dual Agonist Compound Study

The mice were randomized into three study groups (n=10 per group) based on the body weight recorded on Day −1. The groups were: Group 1: Vehicle, BID, SC. Group 2: teduglutide 3.0 mg/kg, BID, SC. Group 3: SEQ ID NO 3, 3.0 mg/kg, BID, SC. Group 4: SEQ ID NO 6, 3.0 mg/kg, BID, SC. Dosing volume was 5 ml/kg, SC. Compounds were dissolved in PBS buffer containing 3% mannitol and 0.6% L-His to obtain a final concentration of 0.6 mg/ml and pH adjusted to 9.0. Animals were dosed with a 27 g×⅝″ needle (Monoject™, Kendall) connected to a 1 ml syringe (Luer-Lock™, Becton). The subcutaneous injections were placed at the lower back. Results on cumulative food intake are listed below in Table 10.

TABLE 10
Cumulative food intake - GLP1R-GLP2R dual agonist compound study
Day 0-7
(g ± S.E.M.)

| | |
|---|---|
| Group 1: | 30.04 ± 1.23 |
| Vehicle | |
| Group 2: | 31.82 ± 6.52 |
| Teduglutide, 3.0 mg/kg (BID) | |
| Group 3: | 26.03 ± 2.49* |
| SEQ ID NO 3, 3.0 mg/kg (BID) | |
| Group 4: | 21.96 ± 1.46*** |
| SEQ ID NO 6, 3.0 mg/kg (BID) | |

Mean ± SEM (n = 10);
*P < 0.05,
**P < 0.01,
***P < 0.001 compared to vehicle;
(One-way ANOVA w/ Dunnett's post hoc test)

### Results:

SEQ ID NO 3 and 6 dosed twice daily for 7 days considerably reduced cumulative food intake compared to vehicle and teduglutide treated controls.

### Example 7

On day 7, an oral glucose tolerance test (OGTT) was performed. Animals were mildly fasted as all food was removed 4 hours prior to the oral glucose load. Vehicle (SC) and compounds were administered subcutaneously (SC) 2 hours prior to the OGTT. At t=0 mice received an oral glucose load (2 g/kg; 4 ml/kg; 500 mg/ml, Fresenius Kabi, Sweden). Glucose was given as gavage via a gastrically-placed tube connected to a syringe ensuring accurate dosing. Blood glucose was measured from tail vein blood before glucose (at t=−120 and 0 min, baseline) and after glucose administration at t=15, 30, 60 and 120 min. Blood samples (tail vein) was collected into 10 μl heparinized glass capillary tubes and immediately suspended in buffer (0.5 ml of glucose/lactate system solution (EKF-diagnostics, Germany) and analyzed for glucose on the test day using a BIOSEN c-Line glucose meter (EKF-diagnostics, Germany) according to manufacturer's instructions. Results from the OGTT are listed below in Table 11.

TABLE 11
Oral glucose tolerance test - GLP1R-GLP2R dual agonist compound study
Day 7
AUC, 0-120 min (mmol/L * min ± S.E.M.)

| | |
|---|---|
| Group 1: | 882 ± 109 |
| Vehicle | |
| Group 2: | 890 ± 182 |
| Teduglutide, 3.0 mg/kg (BID) | |
| Group 3: | 503 ± 97***^{###} |
| SEQ ID NO 3, 3.0 mg/kg (BID) | |
| Group 4: | 523 ± 112***^{###} |
| SEQ ID NO 6, 3.0 mg/kg (BID) | |

Mean ± SEM (n = 10);
*P < 0.05,
**P < 0.01,
***P < 0.001 compared to vehicle;
^{#}P < 0.05,
^{##}P < 0.01,
^{###}P < 0.001 compared to Group 2;
(One-way ANOVA w/ Dunnett's post hoc test)

### Results:

SEQ ID NO 3 and 6 dosed twice daily for 7 days significantly reduced fasting blood glucose compared to vehicle and teduglutide treated controls.

### Example 8

On study day 8, the mice were terminated and the GI tract was removed for determination of the gut wet weight. The gut wet weight results are listed below in Table 12.

TABLE 12
Gut wet weight - GLP1R-GLP2R dual agonist compound study
Day 8
(g ± S.E.M.)

| | |
|---|---|
| Group 1: | 2.2 ± 0.2 |
| Vehicle | |
| Group 2: | 3.0 ± 0.3*** |
| Teduglutide, 3.0 mg/kg (BID) | |
| Group 3: | 3.3 ± 0.2***^{#} |
| SEQ ID NO 3, 3.0 mg/kg (BID) | |
| Group 4: | 3.8 ± 0.3***^{###} |
| SEQ ID NO 6, 3.0 mg/kg (BID) | |

Mean ± SEM (n = 10);
*P < 0.05,
**P < 0.01,
***P < 0.001 compared to vehicle;
^{#}P < 0.05,
^{##}P < 0.01,
^{###}P < 0.001 compared to Group 2;
(One-way ANOVA w/ Dunnett's post hoc test)

### Results:

SEQ ID NO 3 and 6 dosed twice daily for 8 days significantly increased the gut wet weight when compared to vehicle and teduglutide treated controls.

### Example 9

### Effect on Body Weight and Gut Proliferation in Diet Induced Obese (DIO) Sprague-Dawley Rats

Thirty (30) male Sprague-Dawley rats (7 weeks of age) were purchased from Taconic (Denmark) and transferred to the Gubra animal unit. During the acclimatization period, the rats were housed two per cage under a 12:12 light dark cycle (lights on from 04.00-16.00 h) at controlled temperature conditions (22±1° C.; 50±10% relative humidity). Throughout the study the rats had ad libitum access to a two choice diet—regular Altromin 1324 rodent chow (Brogaarden, Denmark) and a paste made from Chocolate spread (Nutella, Ferrero Italy), peanut butter and powdered regular Altromin 1324 rodent chow (Brogaarden, Denmark). The animals were kept on the diet for 65 weeks before experimentation. Animals were switched to single housing during the entire study period (day −6 to 25).

The animals were randomized into three study groups (n=10 per group) based on body weight recorded on day −1 to participate in one of the following groups: Group 1: Vehicle, SC, BID. Group 2: SEQ ID NO 6, 82 nmol/kg (day 2-7), 164 nmol/kg (day 8-14) and 328 nmol/kg (day 15-25), SC, BID. Group 3: Liraglutide, 82 nmol/kg, SC, BID. The vehicle was prepared by dissolving 3% mannitol and 0.6% L-His in phosphate buffer saline and adjusting the pH to 9.0. Dosing took place in the morning (between 06.00 and 08.00 h) and prior to lights out in the afternoon (between 13.30 and 14.00 h). Doses were administered subcutaneously with a dose volume 1 ml/kg. All rats were handled prior to experimentation to acclimatize the animals to handling and procedures. Body weight was measured from day −3 to termination on day 25. Total body fat mass was analyzed before study start (day −1) and before termination (study day 25) by non-invasive EchoMRI-900 (EchoMRI, USA). On day 23 of the study, rats were subjected to an oral glucose tolerance test (OGTT). Animals were mildly fasted as all food was removed 4 hours prior to the oral glucose load. Vehicle and compounds were administered subcutaneously (SC) 2 hours prior to the OGTT. At t=0 rats received an oral glucose load (2 g/kg; 4 ml/kg; 500 mg/nil, Fresenius Kabi, Sweden). Glucose was given as gavage via a gastrically-placed tube connected to a syringe ensuring accurate dosing. Blood glucose was measured from tail vein blood before (t=−120 and 0 min, baseline) and after glucose administration (t=15, 30, 60 and 120 min). Blood samples was collected into 10 μl heparinized glass capillary tubes and immediately suspended in buffer (0.5 ml of glucose/lactate system solution (EKF-diagnostics, Germany) and analyzed for glucose on the test day using a BIOSEN c-Line glucose meter (EKF-diagnostics, Germany) according to manufacturer's instructions. On study day 25, the rats were terminated and the GI tract was removed. Intestines were flushed with saline, weighed and then fixated in 4% PFA for determination of small and large intestinal volume by stereological methods as described by Hansen et al. 2013, Am J Transl Res 5(3): 347-58).

The results on body weight are listed below in Table 13. Results on whole body fat mass are listed in Table 14 while the results of the OGTT are listed in Table 15. Finally, the results on small and large intestinal volume are given in Table 16 and 17.

TABLE 13
Body weight at study start (g ± S.E.M.)
Day 0 and 25

| | Dose | | | | | |
|---|---|---|---|---|---|---|
| | 0 nmol/kg | | 82 nmol/kg | | 328 nmol/kg | |
| | Day 0 | Day 25 | Day 0 | Day 25 | Day 0 | Day 25 |
| Vehicle | 978.3 ± 31.3 | 926.6 ± 30.3 | | | | |
| Liraglutide | | | 984.4 ± 29.4 | 801.5 ± 25.4** | | |
| SEQ ID NO 6 | | | | | 962.7 ± 27.8 | 803.9 ± 35.5* |

Mean ± SEM (n = 7-10);
*P < 0.05,
**P < 0.01 compared to vehicle
(One-way ANOVA w/ Dunnett's post hoc test)

TABLE 14
Whole body fat mass at study start (g ± S.E.M.)
Day 0 and 25

| | Dose | | | | | |
|---|---|---|---|---|---|---|
| | 0 nmol/kg | | 82 nmol/kg | | 328 nmol/kg | |
| | Day −1 | Day 25 | Day −1 | Day 25 | Day −1 | Day 25 |
| Vehicle | 399.5 ± 22.8 | 369.2 ± 20.9 | | | | |
| Liraglutide | | | 392.1 ± 21.6 | 272.7 ± 18.9** | | |
| SEQ ID NO 6 | | | | | 400.3 ± 18.3 | 292.2 ± 25.4* |

Mean ± SEM (n = 7-10);
*P < 0.05,
**P < 0.01 compared to vehicle;
(One-way ANOVA w/ Dunnett's post hoc test)

TABLE 15
Blood glucose during an oral glucose tolerance test.
Area under the curve (AUC) −45 to 240 min.
(mmol/L * min ± S.E.M.)
Day 23

| | Dose | | |
|---|---|---|---|
| | 0 nmol/kg | 82 nmol/kg | 328 nmol/kg |
| Vehicle | 2845 ± 215.2 | | |
| Liraglutide | | 2299 ± 161.4 | |
| SEQ ID NO 6 | | | 1731 ± 77.0*** |

Mean ± SEM (n = 7-10);
***P < 0.001 compared to vehicle;
(One-way ANOVA w/ Dunnett's post hoc test)

TABLE 16
Small intestine volume (cm³ ± S.E.M.)
Day 25

| | Dose | | |
|---|---|---|---|
| | 0 nmol/kg | 82 nmol/kg | 328 nmol/kg |
| Vehicle | 4.67 ± 0.48 | | |
| Liraglutide | | 5.36 ± 0.48 | |
| SEQ ID NO 6 | | | 8.50 ± 0.62*** |

Mean ± SEM (n = 5);
***P < 0.001 compared to vehicle;
(One-way ANOVA w/ Dunnett's post hoc test)

TABLE 17
Large intestine volume (cm³ ± S.E.M.)
Day 25

| | Dose | | |
|---|---|---|---|
| | 0 nmol/kg | 82 nmol/kg | 328 nmol/kg |
| Vehicle | 2.17 ± 0.15 | | |
| Liraglutide | | 2.22 ± 0.21 | |
| SEQ ID NO 6 | | | 2.64 ± 0.17 |

Mean ± SEM (n = 5);
(One-way ANOVA w/ Dunnett's post hoc test)

### Results:

The present data show that 25 days of treatment with the lipidated GLP-1 analog liraglutide in DIO rats leads to a significant decreases in body weight and total body fat mass (Table 13 and 14). In addition to this it is shown that treatment with SEQ ID 06 leads to a similar decreases in body weight and total body fat mass (Table 13 and 14), as well as an improved glucose tolerance (Table 15). In addition to these effects, treatment for 25 days with SEQ ID NO 6 lead to a striking increase in small intestine volume as estimated by stereology (Table 16). Neither treatment had a significant effect on large intestine volume (Table 17).

### Example 10

### Effect on Blood Glucose, HbA1c and Gut Proliferation in Diabetic Db/Db Mice.

A total of forty-six (46) diabetic db/db (BKS.Cg-m+/+Leprdb/J) male mice, 5 weeks old at arrival, was obtained from Janvier (France). The mice were acclimatized for two weeks in their new environment with free access to normal chow (Altromin 1324, Brogaarden A/S, Gentofte, Denmark) and domestic quality tap water. They were housed in groups of n=2 in a light-, temperature-, and humidity-controlled room (12-hour light: 12-hour dark cycle, lights On/Off at 04.00/16.00 h; 22±1° C.; 50±10% relative humidity). During the habituation period animals was accustomed to the experimental paradigm.

Baseline, free fed blood glucose samples were obtained bi-weekly prior to study start (blood was collected from the tail vein) in order to screen for the diabetic state. The 40 animals closest to the mean BG/BW value at day −3 were randomized into 4 groups (n=10/group; the remaining 6 animals was be discarded from the main study): Group 1: Vehicle, BID, SC. Group 2: SEQ ID NO 6, 50 nmol/kg, BID, SC. Group 3: SEQ ID NO 6, 250 nmol/kg, BID, SC. Group 4: Liraglutide, 50 nmol/kg, BID, SC. Vehicle and SEQ ID NO 6 were dissolved in 0.6% L-His and 3% Mannitol in PBS (pH 9.0), while liraglutide was dissolved in 0.1% BSA in PBS (pH 7.4). During day 0-58 of the experiment, animals received a subcutaneous dosing twice daily at 06.00-08.00 hours and again between 02.00-04.00 hours. Dose volume was 5 ml/kg.

On experimental day 56 an oral glucose tolerance test was performed. Animals were mildly fasted as food was removed 4 hours prior to administration of the glucose bolus. Compounds were administered 45 minutes prior to the OGTT. At t=0, mice received an oral glucose load of 2 g/kg glucose (Glucose 200 mg/ml, Fresenius Kabi, Sweden). Glucose was given as gavage via a gastrically placed tube connected to a syringe ensuring accurate dosing. Blood samples were collected from the tail vein and blood-glucose was measured at time points −60, 0, 15, 30, 60, 120 and 240 minutes after the glucose administration. Samples for measuring HbA1c was taken in parallel with blood glucose at t=−60 minutes. Mice were re-fed after the last blood sampling.

At the end of the study period mice were terminated by decapitation under isoflurane anaesthesia. The entire intestine was dissected out and divided into small and large which was then measured in length. Intestines were flushed with saline, weighed and then fixated in 4% PFA for determination of small and large intestinal volume by stereological methods as described by (Hansen et al. 2013, Am J Trans! Res 5(3): 347-58). In addition, termination blood samples were taken and measured for plasma citrulline as described by (Jaisson et al. 2012, Analytical and Bioanalytical Chemistry 402: 1635-41).

The results on blood glucose and HbA1c are listed below in Table 18 and 19. Results of the OGTT are given in Table 20. The stereological volume and surface area estimations on the small and large intestine are listed in Table 21, 22, 23 and 24.

TABLE 18
Blood glucose, fed state (mmol/L ± S.E.M.)
Day 56

| | Dose | | |
|---|---|---|---|
| | 0 nmol/kg | 50 nmol/kg | 250 nmol/kg |
| Vehicle | 15.9 ± 1.8 | | |
| SEQ ID NO 6 | | 7.2 ± 1.0*** | |
| SEQ ID NO 6 | | | 5.9 ± 0.4*** |
| Liraglutide | | 5.6 ± 0.7*** | |

Mean ± SEM (n = 8-10);
***P < 0.001 compared to vehicle;
(One-way ANOVA w/ Dunnett's post hoc test)

TABLE 19
HbA1c (% ± S.E.M.)
Day 56

| | Dose | | |
|---|---|---|---|
| | 0 nmol/kg | 50 nmol/kg | 250 nmol/kg |
| Vehicle | 6.2 ± 0.4 | | |
| SEQ ID NO 6 | | 5.0 ± 0.2** | |
| SEQ ID NO 6 | | | 4.4 ± 0.1*** |
| Liraglutide | | 4.5 ± 0.2*** | |

Mean ± SEM (n = 8-10);
**P < 0.01 and
***P < 0.001 compared to vehicle;
(One-way ANOVA w/ Dunnett's post hoc test)

TABLE 20
Blood glucose during an oral glucose tolerance test.
Area under the curve (AUC) −240 to 240 min.
(mmol/L * min ± S.E.M.)
Day 56

| | Dose | | |
|---|---|---|---|
| | 0 nmol/kg | 50 nmol/kg | 250 nmol/kg |
| Vehicle | 9172 ± 924 | | |
| SEQ ID NO 6 | | 3543 ± 469*** | |
| SEQ ID NO 6 | | | 2472 ± 119***,^{#} |
| Liraglutide | | 3123 ± 278*** | |

Mean ± SEM (n = 8-10);
***P < 0.001 compared to vehicle;
(One-way ANOVA w/ Dunnett's post hoc test)
^{#}P < 0.05 compared to Liraglutide;
(Student's t-test)

TABLE 21
Small intestine volume (mm³ ± S.E.M.)
Day 58

| | Dose | | |
|---|---|---|---|
| | 0 nmol/kg | 50 nmol/kg | 250 nmol/kg |
| Vehicle | 1177 ± 45.1 | | |
| SEQ ID NO 6 | | 2097 ± 72.7*** | |
| SEQ ID NO 6 | | | 2043 ± 62.3*** |
| Liraglutide | | 1072 ± 38.1 | |

Mean ± SEM (n = 8-10);
***P < 0.001 compared to vehicle;
(One-way ANOVA w/ Dunnett's post hoc test)

TABLE 22
Large intestine volume (mm³ ± S.E.M.)
Day 58

Dose

| | 0 nmol/kg | 50 nmol/kg | 250 nmol/kg |
|---|---|---|---|
| Vehicle | 247.5 ± 14.8 | | |
| SEQ ID NO 6 | | 274.2 ± 7.0 | |
| SEQ ID NO 6 | | | 288.2 ± 23.7 |
| Liraglutide | | 232.6 ± 7.0 | |

Mean ± SEM (n = 8-10);
***P < 0.001 compared to vehicle;
(One-way ANOVA w/ Dunnett's post hoc test)

TABLE 23
Small intestine surface area (cm² ± S.E.M.)
Day 58

| | Dose | | |
|---|---|---|---|
| | 0 nmol/kg | 50 nmol/kg | 250 nmol/kg |
| Vehicle | 180.7 ± 12.3 | | |
| SEQ ID NO 6 | | 348.9 ± 10.8*** | |
| SEQ ID NO 6 | | | 344.0 ± 20.8*** |
| Liraglutide | | 144.3 ± 8.4 | |

Mean ± SEM (n = 8-10);
***P < 0.001 compared to vehicle;
(One-way ANOVA w/ Dunnett's post hoc test)

TABLE 24
Large intestine surface area (cm² ± S.E.M.)
Day 58

| | Dose | | |
|---|---|---|---|
| | 0 nmol/kg | 50 nmol/kg | 250 nmol/kg |
| Vehicle | 13.4 ± 1.9 | | |
| SEQ ID NO 6 | | 3.6 ± 1.2 | |
| SEQ ID NO 6 | | | 18.6 ± 1.9 |
| Liraglutide | | 14.1 ± 0.7 | |

Mean ± SEM (n = 8-10);
(One-way ANOVA w/ Dunnett's post hoc test)

### Results:

The present data show that 8 weeks of treatment with liraglutide leads to a reduction in fed-state blood glucose levels, HbA1c levels as well as an improving of glucose tolerance (Tables 18, 19 and 20). Treatment with SEQ ID 06 also leads to a reduction in fed-state blood glucose, HbA1c levels as well as an improving of glucose tolerance in a dose dependent manner. The animals treated with 250 nmol/kg of SEQ ID NO 6 had an even further improved glucose tolerance compared to animals treated with liraglutide (P=0.03, Student's t-test, Table 20).

In addition, 8 weeks of treatment with SEQ ID NO 6 lead to a marked increase in small intestine volume as well as surface area, and here the 50 nmol/kg low dose was equally effective as the 250 nmol/kg high dose (Tables 21 and 23). There was no significant effect on large intestine volume or surface area from any of the compounds (Tables 22 and 24).

### Example 11

### Acute Effect on Food Intake in Lean NMRI Mice.

A total of thirty-five (35) male NMRI mice (4 weeks old, mean BW 28.11 g at the time of arrival) were obtained from Janvier (France) and allocated to the HM2 system. All animals were housed in groups of four. The animal room environment were controlled (targeted ranges: temperature 22±2° C.; relative humidity 50±10%; light/dark cycle: 12 hours light, 12 hours dark, lights off from 14.00 to 02.00 hours). The mice had ad libitum access to Altromin (Brogaarden, Denmark) and tap water. Mice arrived 5 days prior to the initiation of the study and were transferred to the HM-2 system. From arrival day no food was provided in the cages thereby forcing the animals to learn to eat from the food channels thus habituating the animals faster to the HM-2 system. Food intake and body weight were measured from day −3 and throughout the study. Food intake data were collected using the HM-2 real-time food intake monitoring system, an online computerized feeding system using digital weighing cells. As the animals were uniquely identified with microchips, each individual mouse was identified by its corresponding microchip upon entry and exit from the food channel. The HM-2 system continuously and in real-time mode monitored feeding activity in two independent channels and recorded the start and finishing time of each meal and the amount of food consumed by each animal within the selected time interval without human intervention. The net food intake was calculated by the HM-2 control unit software (HMLab, MBRose, Faaborg, Denmark). Animals were randomized at day 0 according to body weight (and mean cumulative food intake from last 24 hours into the following experimental groups (n=8-9): Group 1: Vehicle 0 nmol/kg s.c. Group 2: Liraglutide 50 nmol/kg s.c. Group 3: SEQ ID NO 23 50 nmol/kg s.c.

The first and only dose was administrated at experimental day 0 between 13.30 and 14.00 (just prior to lights off). Doses were administered subcutaneously (dose volume 5 ml/kg), in the lower back region. Food intake was measured up to 68 hours following the dosing of the animals. Cumulative food intake after 12, 24 and 60 hours is found in Table 25.

TABLE 25
Cumulative food intake following administration of a single dose
(grams pr mice ± S.E.M.) 12, 24 and 60 hours after dosing

| | Dose | | | | | |
|---|---|---|---|---|---|---|
| | 0 nmol/kg | | | 50 nmol/kg | | |
| | 12 h | 24 h | 60 h | 12 h | 24 h | 60 h |
| Vehicle | 3.84 ± 0.44 | 4.61 ± 0.39 | 12.69 ± 3.08 | | | |
| Liraglutide | | | | 1.72 ± 0.20*** | 2.62 ± 0.27*** | 12.83 ± 1.78 |
| SEQ ID NO 23 | | | | 1.82 ± 0.24*** | 2.83 ± 0.22*** | 11.43 ± 1.97 |

Mean ± SEM (n = 8-9);
***P < 0.001 compared to vehicle;
(One-way ANOVA w/ Dunnett's post hoc test)

### Results:

The present data show that treatment with liraglutide significantly reduced the acute food intake for up to 24 hours following administration of a single dose of 50 nmol/kg in mice (Table 25). Furthermore, it shows that a single, equally low, dose of SEQ ID NO 23 is also able to significantly reduce acute food intake to an similar extend as liraglutide in lean NMRI mice both after 12 and 24 hours. At 60 hours liraglutide show no difference in food intake compared to the vehicle, whereas SEQ ID NO 23 tends to still have an effect.

### Example 12

### Sub-Acute Effect on Body Weight in Lean CS7BL/6J Mice.

Eighteen (18) male C57/BL6J mice obtained from Janvier, France, were used. At the time of experimentation the mice had reached an age of 9 weeks. The mice were acclimatized for one week in their new environment and offered regular chow diet (Altromin 1324, Brogaarden A/S, Denmark) and domestic quality tap water. Animals were housed six per cage during the study, in a light-, temperature-, and humidity-controlled room (12-hour light:12-hour dark cycle, lights on/off at 4 AM/4 PM hour; 22±1° C.; 50±10% relative humidity).

Mice were randomized into the following seven study groups based on the body weight recorded on day −3: Group 1: Vehicle, BID, SC, Group 2: SEQ ID NO 23, 50 nmol/kg BID, SC. Dosing volume was 5 ml/kg, SC.

The compounds were dissolved in PBS buffer containing 0.1% BSA, pH 7.4. The subcutaneous injections were placed at the lower back. All mice were handled for 3 days prior to experimentation to acclimatize the animals to handling and injections. Body weight is measured from day −3 to day 7 and the percent change to baseline body weight is shown in Table 26.

TABLE 26
Body weight change in percent to baseline body weight (% ± S.E.M.)
Day 0, 2, 5 and 7.

| | Dose | | | | | | | |
|---|---|---|---|---|---|---|---|---|
| | 0 nmol/kg | | | | 50 nmol/kg | | | |
| | Day | | | | | | | |
| | 0 | 2 | 5 | 7 | 0 | 2 | 5 | 7 |
| Vehicle | 100.0 ± 0.0 | 97.8 ± 1.2 | 95.5 ± 1.4 | 100.0 ± 1.4 | | | | |
| SEQ ID NO 23 | | | | | 100.0 ± 0.0 | 90.7 ± 0.8*** | 89.5 ± 1.8# | 90.8 ± 1.4*** |

Mean ± SEM (n = 6);
***P < 0.001 compared to vehicle day 2;
#P < 0.05 compared to vehicle day 5;
(One-way ANOVA w/ Dunnett's post hoc test)

The present data show that treatment with 50 nmol/kg SEQ ID NO 23 significantly reduced the body weight in lean C57BL/63 mice after two days of administration. This body weight loss was sustained for up to seven days of dosing (Table 26).

1. A pharmaceutical composition comprising a first peptide or a lipidated analog thereof, said first peptide providing agonist activity towards the human GLP-1 receptor and a second peptide or a lipidated analog thereof, said second peptide providing agonist activity towards the human GLP-2 receptor, wherein the relative agonist activity of the peptides towards the human GLP-1 receptor (GLP-1Rᵣₑₗₐₜᵢᵥₑ) is at least 0.01, and wherein the relative agonist activity of the peptides towards the human GLP-2 receptor (GLP-2Rᵣₑₗₐₜᵢᵥₑ) is at least 0.01, and wherein (GLP-1Rᵣₑₗₐₜᵢᵥₑ)(GLP-2Rᵣₑₗₐₜᵢᵥₑ) is at least 0.01, for use in the induction of gut proliferation.

2. A pharmaceutical composition comprising a first peptide or a lipidated analog thereof, said first peptide providing agonist activity towards the human GLP-1 receptor and a second peptide or a lipidated analog thereof, said second peptide providing agonist activity towards the human GLP-2 receptor, wherein the relative agonist activity of the peptides towards the human GLP-1 receptor (GLP-1Rᵣₑₗₐₜᵢᵥₑ) is at least 0.01, and wherein the relative agonist activity of the peptides towards the human GLP-2 receptor (GLP-2Rᵣₑₗₐₜᵢᵥₑ) is at least 0.01, and wherein the peptides provides a (GLP-2Rᵣₑₗₐₜᵢᵥₑ)>(GLP-1Rᵣₑₗₐₜᵢᵥₑ).

3. A peptide having dual agonist activity towards the human GLP-1 receptor and the human GLP-2 receptor, wherein the relative agonist activity of the dual peptide towards the human GLP-1 receptor (GLP-1Rᵣₑₗₐₜᵢᵥₑ) is at least 0.01, and wherein the relative agonist activity of the dual peptide towards the human GLP-2 receptor (GLP-2Rᵣₑₗₐₜᵢᵥₑ) is at least 0.01, and wherein (GLP-1Rᵣₑₗₐₜᵢᵥₑ)(GLP-2Rᵣₑₗₐₜᵢᵥₑ) is at least 0.01, a pharmaceutically acceptable salt, solvate or lipidated analog thereof.

4. Composition according to claim 1, wherein GLP-2Rᵣₑₗₐₜᵢᵥₑ is at least 0.1, preferably at least 0.2, more preferably at least 0.3, even more preferably at least 1.

5. Composition according to claim 1, wherein (GLP-Rᵣₑₗₐₜᵢᵥₑ)(GLP-2Rᵣₑₗₐₜᵢᵥₑ) is at least 0.02, preferably at least 0.03, more preferably at least 0.2, even more preferably at least 0.5.

6. Composition according claim 1, wherein (GLP-2Rᵣₑₗₐₜᵢᵥₑ)>(GLP-1Rᵣₑₗₐₜᵢᵥₑ), preferably wherein (GLP-2Rᵣₑₗₐₜᵢᵥₑ)>2(GLP-1Rᵣₑₗₐₜᵢᵥₑ), even more preferably wherein (GLP-2Rᵣₑₗₐₜᵢᵥₑ)>10(GLP-1Rᵣₑₗₐₜᵢᵥₑ).

7. Peptide comprising a sequence according to SEQ ID NO: 1 or an lipidated peptide analog having no more than 2 deviations in the amino acid sequence of SEQ ID NO: 1;
H-X₂-D-G-X₅-F-X₇-X₈-X₉-X₁₀-S-X₁₂-Y-X₁₄-X₁₅-X₁₆-L-A-X₁₉-X₂₀-X₂₁-F-I-X₂₄-W-L-X₂₇-X₂₈-X₂₉-X₃₀-X₃₁-X₃₂-X₃₃, wherein
X₂ is Gly, Ala, Aib, Sar;
X₅ is Thr, Ser;
X₇ is Thr, Ser;
X₈ is Thr, Asp, Ser, Glu;
X₉ is Asp, Glu;
X₁₀ is Leu, Nle, Met, Val, Tyr;
X₁₂ is Thr, Ser, Ala;
X₁₄ is Leu, Nle, Met, Val;
X₁₅ is Asp, Glu;
X₁₆ is Ala, Asn, Gln, Gly, Ser, Glu, Asp, Arg, Lys;
X₁₉ is Ala, Val, Leu;
X₂₀ is Arg, Lys, His;
X₂₁ is Asp, Glu;
X₂₄ is Ala, Asn, Asp, Gln, Glu, Lys, Arg;
X₂₇ is Ile, Leu, Val, Lys, Arg, Nle;
X₂₈ is Gln, Asn, Lys, Arg;
X₂₉ is Thr, Ser, Lys, Arg;
X₃₀ is Lys, Arg;
X₃₁ is Ile or absent;
X₃₂ is Thr or absent;
X₃₃ is Asp or absent,
or a pharmaceutically acceptable salt or solvate thereof.

8. Peptide or lipidated peptide analog according to claim 7, wherein X₇ is Thr, X₉ is Glu and X₁₉ is Ala.

9. Peptide or lipidated peptide analog according to claim 8, comprising a peptide sequence according to SEQ ID NO: 2
R¹-H-G-D-G-S-F-T-X₈-E-X₁₀-S-T-Y-L-D-X₁₆-L-A-A-R-D-F-I-X₂₄-W-L-I-Q-T-K-X₃₁-X₃₂-X₃₃-R², wherein
X₈ is Thr, Asp, Ser, Glu;
X₁₀ is Leu, Nle, Met, Val, Tyr;
X₁₆ is Ala, Asn, Gin, Gly, Ser, Glu, Asp, Arg, Lys;
X₂₄ is Ala, Asn, Asp, Gln, Glu, Lys, Arg;
X₃₁ is Ile or absent;
X₃₂ is Thr or absent;
X₃₃ is Asp or absent;
and wherein R¹ is hydrogen, methyl, acetyl, formyl, benzoyl, trifluoroacetyl, and R² is NH₂ or OH.

10. Peptide or lipidated peptide analog according to claim 7, wherein X₈ is Asp or Ser, preferably Ser; X₁₀ is Leu or Nle; X₁₆ is Ala or Asn; X₂₄ is Ala or Asn; and wherein X₃₁, X₃₂, and X₃₃ are Ile, Thr and Asp or wherein X₃₁, X₃₂, and X₃₃ are absent

11. Peptide or lipidated peptide analog according to claim 7, the peptides being selected among
(SEQ ID NO: 3)
R¹-H-G-D-G-S-F-T-D-E-L-S-T-Y-L-D-A-L-A-A-R-D-F-I-A-
L-I-Q-T-K-R²;
(SEQ ID NO: 4)
R¹-H-G-D-G-S-F-T-S-E-L-S-T-Y-L-D-A-L-A-A-R-D-F-I-N-
W-L-I-Q-T-K-R²;
(SEQ ID NO: 5)
R¹-H-G-D-G-S-F-T-D-E-L-S-T-Y-L-D-N-L-A-A-R-D-F-I-A-
W-L-I-Q-T-K-I-T-D-R²;
(SEQ ID NO: 6)
R¹-H-G-D-G-S-F-T-S-E-L-S-T-Y-L-D-A-L-A-A-R-D-F-I-A-
W-L-I-Q-T-K-I-T-D-R²;
and
(SEQ ID NO: 7)
R¹-H-G-D-G-S-F-T-S-E-L-S-T-Y-L-D-N-L-A-A-R-D-F-I-N-
W-L-I-Q-T-K-I-T-D-R².

12. Lipidated peptide analog according to claim 7, comprising one lipidated amino acid residue.

13. Peptide according to claim 12, wherein the lipidated amino acid residue is present at any one of the positions X₇-X₁₉.

14. Peptide according to claim 13, wherein the lipidated amino acid residue is present at any one of the positions X₁₂, X₁₄, X₁₆ and X₁₇.

15. Peptide according to claim 14, wherein the lipidated amino acid residue is present at any one of the positions X₁₄ or X₁₇.

16. Peptide according to claim 12, being selected among
(SEQ ID NO: 19)
R¹-H-G-D-G-S-F-[K(C16-yE-)]-S-E-L-S-T-Y-L-D-A-L-A-
A-R-D-F-I-A-W-L-I-Q-T-K-I-T-D-R²;
(SEQ ID NO: 20)
R¹-H-G-D-G-S-F-T-[K(C16-yE-)]-E-L-S-T-Y-L-D-A-L-A-
A-R-D-F-I-A-W-L-I-Q-T-K-I-T-D-R²;
(SEQ ID NO: 21)
R¹-H-G-D-G-S-F-T-S-E-L-[K(C16-yE-)]-T-Y-L-D-A-L-A-
A-R-D-F-I-A-W-L-I-Q-T-K-I-T-D-R²;
(SEQ ID NO: 22)
R¹-H-G-D-G-S-F-T-S-E-L-S-[K(C16-yE-)]-Y-L-D-A-L-A-
A-R-D-F-I-A-W-L-I-Q-T-K-I-T-D-R²;
(SEQ ID NO: 23)
R¹-H-G-D-G-S-F-T-S-E-L-S-T-Y-[K(C16-yE-)]-D-A-L-A-
A-R-D-F-I-A-W-L-I-Q-T-K-I-T-D-R²;
(SEQ ID NO: 24)
R¹-H-G-D-G-S-F-T-S-E-L-S-T-Y-L-D-[K(C16-yE-)]-L-A-
A-R-D-F-I-A-W-L-I-Q-T-K-I-T-D-R²;
(SEQ ID NO: 25)
R¹-H-G-D-G-S-F-T-S-E-L-S-T-Y-L-D-A-[K(C16-yE-)]-A-
A-R-D-F-I-A-W-L-I-Q-T-K-I-T-D-R²;
and
(SEQ ID NO: 26)
R¹-H-G-D-G-S-F-T-S-E-L-S-T-Y-L-D-A-L-A-A-
[K(C16-yE-)]-D-F-I-A-W-L-I-Q-T-K-I-T-D-R².

17. Peptide according to claim 16, being selected among
(SEQ ID NO: 23)
R¹-H-G-D-G-S-F-T-S-E-L-S-T-Y-[K(C16-yE-)]-D-A-L-A-
A-R-D-F-I-A-W-L-I-Q-T-K-I-T-D-R²;
and
(SEQ ID NO: 25)
R¹-H-G-D-G-S-F-T-S-E-L-S-T-Y-L-D-A-[K(C16-yE-)]-A-
A-R-D-F-I-A-W-L-I-Q-T-K-I-T-D-R²;
and

18. Composition or peptide according to claim 2 for use in the treatment or prophylactic treatment of a human or animal subject.

19. Composition or peptide for use according to claim 18, said treatment or prophylactic treatment being the treatment or prophylactic treatment of a condition related to gut and brain relates diseases or metabolic disorders, such as gastrointestinal inflammation, short bowel syndrome and Crohn's disease.

20. Composition or peptide for use according to claim 18, said treatment or prophylactic treatment being the treatment or prophylactic treatment of non-alcoholic steatohepatitis (NASH).

21. Composition or peptide for use according to claim 18, said treatment or prophylactic treatment being the treatment or prophylactic treatment of surgical trauma.

22. Pharmaceutical or veterinary composition comprising a peptide according to claim 3, and at least one pharmaceutical or veterinary excipient.

23. Nucleic acid molecule comprising a nucleic acid sequence encoding the peptide of claim 3.

24. Method of producing a peptide, wherein the method comprising a step of providing expression of the nucleic acid molecule comprising a nucleic acid sequence encoding the peptide of claim 3 and purifying the product thus produced.
